SEQUENCE LISTING (17 sequences)

SEQ ID NO: 1 - protein, 363 aa, Homo sapiens
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - protein, 367 aa, Macaca mulatta
PGARKLRAVLEKLRLSHQDISKAAKVVNGVVGHLLRRLKCEPEFGGVEQLHTGSYYEHVKISAPNEFDVMFKLQVPRIQLEEYSDTGAYYFVKFKRNPEGNPLSLFLEDEILSASKMLSKFRKIIKEEINNIKDVQPDADVIMKRKRPGSPAVTLLINKEISVDITLALELKTSWPASTQEGLAIKNWLSAKVRQQLRLKPFYLVPKHAKEGNGFQEETWRLSFSHIEKEILSNHGKSKTCCENKEMKCCRKDCLKLMKYLLEQLKEKFKDKKHLDKFSSYHVKTAFFHLCTENPQDSQWDPKDLGLCFDNCVTYFLECLRTEQLKNYFIPGFNLFSSNLIDKTSKEFLSKQIEYERNNEFPVFGEF

SEQ ID NO: 3 - protein, 363 aa, Papio anubis
PGARKLRAVLEKLRLSHQDISKAAKVVNGVVGHLLHRLKCESEFEGVEQLHTGSYYEHVKISAPNEFDVMFKLQVPRIQLEEYSDTGAYYFVKFKRNPKGNPLSQFLEDEILSASKMLSQFRKIIKEEINNIKDTDVIMKRKRRGSPAVTLLINKEISVDITLALELKTSWPASTQEGLAIKNWLSAKVRQQLRLKPFYLVPKHAKEGNGFQEETWRLSFSHIEKEILSNHGKSKTCCENKEMKCCRKDCLKLMKYLLEQLKEKFKDKKHLDKFSSYHVKTAFFHLCTENPQDSQWDPKDLGLCFDNCVTHFLQCLRTEKLANYFIPGFNLFSSNLIDKSSKEFLSKQIEYERNNEFPVFGEF

SEQ ID NO: 4 - protein, 363 aa, Nomascus leucogenys
PGARKLRAVLEKLKLSRQEISEAAEVVNGVVDHLLRRLQKCDSEFRGVGLLRTGSYYEHVKISAPNEFDVMFKLEVPRIQLEEYSNTGAYYFVKFKRNPKENPLSQFLEGEILSASKMLSKFRKIIKEEINNIKDTDVIMKRKRGGSPAVTLLISEKISVDITLALESNSNSWPASTQEGLPIKDWLSGKVRRQLRLKPFYLVPKHAKEGNGFQEETWRLSFSHIEKEILNNHGKTKTCCENKNVKCCRKDCLKLMKYLLEQLKERFKDKKHLDKFSSYHVKTAFFHVCTENPQDSQWDPKNLGFCFDKCVIYFLKCLRTEQLGNYFIPGFNLFSSNLIDKRSKEFLSKQIEYERNNEYPVFG

SEQ ID NO: 5 - protein, 365 aa, Hylobates lar
PGARKLPAVLEKLKLSRQEICEAAEVVNGVVDHLLRRLQKCDSEFRGVGLLRTGSYYEHVKISAPNEFDVMFKLEVPRIQLEEYSNTGAYYFVKFKRNPKENPLCQFLEGEILSASKMLSKFRKIIKEEINNIKDTDVILKRKRGGSPAVTLLISEKISVDITLALESKSSSWPASTQEGLPIKDWLSGKVRTQLRLKPFYLVPKHAKEGNGFQEETWRLSFSHIEKEILNNHGETKTCCENKKVKCCRKDCLKLMKYLLEQLKERFKDKKHLDKFSSYHVKTAFFHVCTENPKDSQWDPKDLGLCFDKCVIYFLQCLSTEQLGNYFIPGFNLFSSNLIDKRSKEFLSKQIEYERNNEYPVFGKC

SEQ ID NO: 6 - protein, 364 aa, Pongo abelii
PGASKLRAVLEKLKLSRLEISKAAEVVNRVVDHLLRRLQKYNSEFRGVGLLRTGSYYEHVKISAPNEFDVMFKLEVPRIQLEEYSNTGAYYFVKFKRNPKENPLSQFLEGEILSASKMLSKFRKIIKEEINNIKDTDVIMKRKRGGSPAVTLLISEEISVDITLALESKSSWPASTQEGLPIKNWLSAKVRKQLRLKPFYLVPKHAKEGNGFQEETWRLSFSHIEKEILNNHGKSKTCCEDKEVKCCRKDCLKLMKYLLEQLKERFKDKKHLDKFSSYHVKTAFFHVCTQNPQDSQWDPKDLGLCFDNCVTYFLQCLRTEQLGNYFIPGFNLFSSNLIDKRSKEFLTKQIEYERNNEFPVFDEF

SEQ ID NO: 7 - protein, 361 aa, Pan troglodytes
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINNIKDVIMKRKRGGSPAVTLLISEKISVDITLALESKSSWPASTQEGLPIKNWLSAKVRKQLRLKPFYLVPKHAKEGNGFQEETWRLSFSHIEKEILNNHGKSKTCCEIKEAKCCRKDCLKLMKYLLEQLKEKFKDKKHLDKFSSYHVKTAFFHVCTENPQDSQWDRKDLGLCFDNCVTYFLQCLRIEKLENYFIPEFNLFSSDLIDKRSKEFLTKQIEYERNNEFPVFDEF

SEQ ID NO: 8 - protein, 363 aa, Gorilla gorilla
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINNIKDTDVIMKRKRGGSPAVTLLISEKISVDITLALESKSSWPASTQEGLRIQNWLSAKVRRQLRLKPFYLVPKHAKEGNGFQEETWRLSFSHIEKEILNNHGKSKTCCENKEAKCCRKDCLKLMKYLLEQLKERFKDKKHLDKFSSYHVKTAFFHVCTQNPQDSQWDRKQLGLCFDNCVTYFLQCLRTERLENYFIPEFNLFSRNLIDKRSKEFLTKQIEYERNNEFPVFDEF

SEQ ID NO: 9 - protein, 369 aa, synthetic construct
PGAXKLXAVLEKLXLSXXXIXXAAXXVXXVVXHLLXRLXXXXXXFXGVXXLXTGSYYEHVKISAPNEFDVMFKLXVPRIQLEEYSXTXAYYFVKFKRNPXXNPLXXFLEXEILSASKMLSXFRKIIKEEINXIKXXXXXXDVIXKRKRXGSPAVTLLIXXXISVDITLALEXXXXSWPASTQEGLXIXXWLSXKVRXQLRLKPFYLVPKHAKEGNGFQEETWRLSFSHIEKEILXNHGXXKTCCEXKXXKCCRKDCLKLMKYLLEQLKEXFKDKKHLDKFSSYHVKTAFFHXCTXNPXDSQWDXKXLGXCFDXCVTXFLXCLXXEXLXNYFIPXFNLFSXXLIDKXSKEFLXKQIEYERNNEXPVFXXX

SEQ ID NO: 10 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 11 - protein, 159 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAA

SEQ ID NO: 12 - protein, 21 aa, Thosea asigna virus
  VARIANT 1..3: note = can be present or absent
GSGEGRGSLLTCGDVEENPGP

SEQ ID NO: 13 - protein, 22 aa, Porcine teschovirus 1
  VARIANT 1..3: note = can be present or absent
GSGATNFSLLKQAGDVEENPGP

SEQ ID NO: 14 - protein, 23 aa, Equine rhinitis A virus
  VARIANT 1..3: note = can be present or absent
GSGQCTNYALLKLAGDVESNPGP

SEQ ID NO: 15 - protein, 25 aa, Foot-and-mouth disease virus
  VARIANT 1..3: note = can be present or absent
GSGVKQTLNFDLLKLAGDVESNPGP

SEQ ID NO: 16 - protein, 8 aa, Gallus gallus
SIINFEKL

SEQ ID NO: 17 - DNA, 1098 bp, synthetic construct
  source 1..1098: mol_type = other DNA
atgcccggcgccagcaagctgagggccgtgctggagaagctgaagctgagcagggacgacatcagcaccgccgccggcatggtgaagggcgtggtggaccacctgctgctgaggctgaagtgcgacagcgccttcaggggcgtgggcctgctgaacaccggcagctactacgagcacgtgaagatcagcgcccccaacgagttcgacgtgatgttcaagctggaggtgcccaggatccagctggaggagtacagcaacaccagggcctactacttcgtgaagttcaagaggaaccccaaggagaaccccctgagccagttcctggagggcgagatcctgagcgccagcaagatgctgagcaagttcaggaagatcatcaaggaggagatcaacgacatcaaggacaccgacgtgatcatgaagaggaagaggggcggcagccccgccgtgaccctgctgatcagcgagaagatcagcgtggacatcaccctggccctggagagcaagagcagctggcccgccagcacccaggagggcctgaggatccagaactggctgagcgccaaggtgaggaagcagctgaggctgaagcccttctacctggtgcccaagcacgccaaggagggcaacggcttccaggaggagacctggaggctgagcttcagccacatcgagaaggagatcctgaacaaccacggcaagagcaagacctgctgcgagaacaaggaggagaagtgctgcaggaaggactgcctgaagctgatgaagtacctgctggagcagctgaaggagaggttcaaggacaagaagcacctggacaagttcagcagctaccacgtgaagaccgccttcttccacgtgtgcacccagaacccccaggacagccagtgggacaggaaggacctgggcctgtgcttcgacaactgcgtgacctacttcctgcagtgcctgaggaccgagaagctggagaactacttcatccccgagttcaacctgttcagcagcaacctgatcgacaagaggagcaaggagttcctgaccaagcagatcgagtacgagaggaacaacgagttccccgtgttcgacgagttctaatga

(19) United States
(12) Patent Application Publication
(10) Pub. No.: US 2025/0255988 A1
(43) Pub. Date: Aug. 14, 2025

(54) MRNA ENCODING A CONSTITUTIVELY-ACTIVE CYCLIC GMP-AMP SYNTHASE AND LIPID DELIVERY VEHICLES FOR SAME

(71) Applicant: Corner Therapeutics, Inc., Watertown, MA (US)

(72) Inventors: Emily GOSSELIN, West Roxbury, MA (US); Dania ZHIVAKI, West Roxbury, MA (US); Jonathan CHOW, Newton, MA (US); Anastasia NIKIFOROV, Watertown, MA (US); Debrup SENGUPTA, Mansfield, MA (US)

(73) Assignee: Corner Therapeutics, Inc., Watertown, MA (US)

(21) Appl. No.: 18/864,462

(22) PCT Filed: May 12, 2023
(86) PCT No.: PCT/US2023/066974

§ 371 Date: Nov. 8, 2024

Related U.S. Application Data
(60) Provisional application No. 63/341,987, filed on May 13, 2022.

(51) Int. Cl. A61K 48/00; C12N 9/12; C12N 15/88

(52) U.S. Cl. CPC A61K 48/0058; C12N 9/1241; C12N 15/88; C12Y 207/07

(57) ABSTRACT

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising a pathogen recognition receptor agonist.

### CROSS-REFERENCE TO RELATED APPLICATIONS

This application claims priority to and benefit of U.S. Provisional Patent Application No. 63/341,987, filed May 13, 2022, which is incorporated herein by reference in its entirety.

### REFERENCE TO AN ELECTRONIC SEQUENCE LISTING

The content of the electronic sequence listing (165532000540SEQLIST.xml; Size: 22,326 bytes; and Date of Creation: May 10, 2023) is herein incorporated by reference in its entirety.

### FIELD

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising a pathogen recognition receptor agonist.

### BACKGROUND

Advances in mRNA chemistry and delivery systems have enabled the rapid production of several effective mRNA COVID-19 vaccines (Hou et al., Nature Review Materials, 6:1078-1094, 2021). However, a recent report found that T cell reactivity to the SARS-CoV-2 spike protein is considerably lower in vaccinated, uninfected individuals than in individuals with a prior SARS-CoV-2 infection (Naranbhai et al., Cell, 185:1-11, 2022). This is concerning given that higher levels of SARS-CoV-2-reactive CD4+ and CD8+ T cell immune responses were found to be associated with milder COVID-19 disease (Rydyznski Moderbacher et al., Cell, 183(4):996-1012, 2020).

As such, formulations for enhancing cellular immune responses elicited by mRNA vaccines are needed in the art. In particular, adjuvants suitable for increasing adaptive immune responses to nucleic acid-encoded antigens are desirable.

### BRIEF SUMMARY

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising a pathogen recognition receptor agonist.

### BRIEF DESCRIPTION OF THE DRAWINGS

FIG. 1 is an alignment of amino acid sequences of primate cGASAN proteins including: human (Homo sapiens, SEQ ID NO:1); Rhesus monkey (Macaca mulatta, SEQ ID NO:2); olive baboon (Papio anubis, SEQ ID NO:3); northern white-cheeked gibbon (Nomascus leucogenys, SEQ ID NO:4); common gibbon (Hylobates lar, SEQ ID NO:5); Sumatran orangutan (Pongo abelii, SEQ ID NO:6); chimpanzee (Pan troglodytes, SEQ ID NO:7); western gorilla (Gorilla gorilla, SEQ ID NO: 8); and a cGASAN consensus sequence (SEQ ID NO:9).

FIG. 2 is a cartoon depicting activation of the stimulator of interferon genes (STING) innate immune signaling pathway by a cyclic GAMP-AMP synthase (cGAS) that has been engineered to be constitutively-active by removal of its amino-terminal phosphoinositide-binding domain. cGASAN localizes to mitochondria where it binds DNA resulting in the production of cGAMP.

FIG. 3A-3C depict characteristics of lipid nanoparticles (LNPs) produced using GenVoy ILM™ ionizable lipid mix (Precision NanoSystems). FIG. 3A shows that LNPs loaded with either OVA, GFP, or cGASAN mRNAs are all less than 200 nm in diameter. FIG. 3B shows that LNPs loaded with either OVA, GFP, or cGASAN mRNAs are all relatively uniform in size, with all exhibiting a polydispersity index of less than 0.3. FIG. 3C shows that LNPs can be loaded with OVA, GFP, or cGASAN mRNA with similar levels of efficiency.

FIG. 4A-4C show that murine dendritic cells (DCs) secrete inflammatory cytokines stimulated by the cGAS-STING pathway in response to treatment with LNPs loaded with mRNA encoding cGASΔN (cGASΔN LNPs). FIG. 4A shows that bone marrow-derived dendritic cells (BMDCs) stimulated with cGASΔN LNPs produce RANTES at significantly higher levels than cells treated with empty LNPs, or only LNPs loaded with mRNA encoding a model antigen. FIG. 4B shows that treatment of BMDCs with cGASΔN LNPs results in significantly higher levels of IP-10 secretion than treatment with empty LNPs, or only LNPs loaded with mRNA encoding a model antigen. FIG. 4C shows that BMDCs treated with cGASΔN LNPs produce IFNα at significantly higher levels than cells treated with empty LNPs, or only LNPs loaded with mRNA encoding a model antigen. X-axis labels for all three plots are as shown for FIG. 4C. P<0.05*, p<0.01**, p<0.001***, p<0.0001****. Statistics were completed using one-way ANOVA with a Tukey post test for multiple comparisons. Data is representative of two experiments.

FIG. 5A-5F show that treatment with LNPs loaded with mRNA encoding cGASΔN (cGASΔN LNPs) activates murine DCs resulting in increased expression of multiple cell surface markers. DCs were found to express significantly higher levels of co-stimulatory molecules CD40 (FIG. 5A), CD86 (FIG. 5B), and CD69 (FIG. 5C) as shown by increases in mean fluorescence intensity (MFI) following treatment with cGASΔN LNPs as compared to treatment with empty LNPs or only LNPs loaded with mRNA encoding a model antigen. DCs were also found to express significantly higher levels of antigen presentation molecules MHC-II (FIG. 5D) and H2kB (MHC-I) (FIG. 5E) when treated with cGASΔN LNPs as compared to treatment with empty LNPs or only LNPs loaded with mRNA encoding a model antigen. Additionally, DCs were found to have higher levels of expression of the CCR7 migratory molecule (FIG. 5F) when treated with cGASΔN LNPs as compared to treatment with empty LNPs or only LNPs loaded with mRNA encoding a model antigen. X-axis labels for all six plots are as shown for FIG. 5E and FIG. 5F. P<0.05*, p<0.01**, p<0.001***, p<0.0001****. Statistics were completed using one-way ANOVA with a Tukey post test for multiple comparisons. Data is representative of two experiments.

FIG. 6A-6C show that LNPs made from different lipid formulations and loaded with different mRNAs exhibit similar characteristics. FIG. 6A shows that LNPs made with GenVoy ILM™ ionizable lipid mix (Precision NanoSystems) or with the custom lipid mix are all less than 150 nm in diameter when loaded with OVA, GFP, or cGASΔN mRNA. FIG. 6B shows that LNPs made with GenVoy ILM™ or the custom lipid mix are all relatively uniform in size, with a polydispersity index less than 0.3. FIG. 6C shows that LNPs made with GenVoy ILM™ or the custom lipid mix allow for efficient encapsulation of OVA, GFP, and cGASΔN mRNAs. Data are representative of three experiments.

FIG. 7A-7F show that cGASΔN mRNA-containing LNPs activate cGAS-STING inflammatory pathways in human monocyte-derived dendritic cells (moDCs). Human moDCs were cultured with LNPs loaded with OVA, GFP, cGASΔN mRNA or a combination of LNPs for 24 hrs, after which supernatants were collected and cytokine secretion was assessed. Human moDCs treated with LNPs loaded with cGASΔN mRNA produced significantly more IL-6 (FIG. 7A), more TNFα (FIG. 7B), and significantly more IP-10 (FIG. 7C) than moDCs treated with LNPs loaded only with mRNA encoding a model antigen. In addition, moDCs secreted significantly more IFNλ1 (FIG. 7D) significantly more IFNβ (FIG. 7E), and more IFNα2 (FIG. 7F) when treated with LNPs loaded with cGASΔN mRNA than moDCs treated with LNPs loaded only with mRNA encoding a model antigen. X-axis labels for all six plots are as shown for FIG. 7E and FIG. 7F. P<0.05*, p<0.01**, p<0.0001****. Statistics were completed using two-way ANOVA with a Tukey post test for multiple comparisons. Data is representative of two experiments. Circles show control conditions devoid of LNPs. Squares show conditions including LNPs formed using GenVoy ILM™. Diamonds show conditions including LNPs formed using the custom lipid mix. White squares and white diamonds show conditions including cGASΔN mRNA-containing LNPs formed using GenVoy ILM™ and the custom lipid mix, respectively.

FIG. 8A-8D show that treatment with cGASΔN mRNA-containing LNPs activates human moDCs. Human moDCs were cultured with LNPs loaded with OVA, GFP, cGASΔN mRNA, or a combination of LNPs for 24 hrs, after which cell surface marker expression was measured. CD83 (FIG. 8A) expression significantly increased when moDCs were treated with LNPs loaded with cGASΔN mRNA as compared to treatment with LNPs loaded only with mRNA encoding a model antigen. Likewise, expression of T cell costimulatory molecules CD40 (FIG. 8B) and CD80 (FIG. 8C) significantly increased when moDCs (both overall population and at the single cell level) were treated with LNPs loaded with cGASΔN mRNA as compared to treatment with LNPs loaded only with mRNA encoding a model antigen. Additionally, MHC Class II (HLA-DR) (FIG. 8D) expression was increased when moDCs were treated with cGASΔN mRNA-containing LNPs as compared to treatment with LNPs loaded only with mRNA encoding a model antigen. P<0.05*, p<0.01**. Statistics completed using two-way ANOVA with a Tukey post test for multiple comparisons. Data is representative of two experiments. Circles show control conditions devoid of LNPs. Squares show conditions including LNPs formed using GenVoy ILM™. Diamonds show conditions including LNPs formed using the custom lipid mix. White squares and white diamonds show conditions including cGASΔN mRNA-containing LNPs formed using GenVoy ILM™ and the custom lipid mix, respectively.

FIG. 9A-9B show that cGASΔN mRNA-containing LNPs activate inflammatory pathways in a human leukemia monocytic cell line (THP-1 cells). Briefly, THP-1 cells were cultured with LNPs loaded with OVA, GFP, cGASΔN mRNA, or a combination of LNPs for 24 hrs, after which supernatants were collected for measurement of cytokine secretion, and cells were collected for assessment of cell surface molecule expression. THP-1 cells treated with cGASΔN mRNA-containing LNPs secreted significantly more IP-10 (FIG. 9A) than THP-1 cells treated with LNPs loaded only with mRNA encoding a model antigen. CD40 expression (FIG. 9B) on THP-1 cells increased after treatment with cGASΔN mRNA-containing LNPs as compared to LNPs loaded only with mRNA encoding a model antigen. P<0.01**, p<0.0001****. Statistics were completed using two-way ANOVA with a Tukey post test for multiple comparisons. Data is representative of two experiments. Circles show control conditions devoid of LNPs. Squares show conditions including LNPs formed using GenVoy ILM™ Diamonds show conditions including LNPs formed using the custom lipid mix. White squares and white diamonds show conditions including cGASΔN mRNA-containing LNPs formed using GenVoy ILM™ and the custom lipid mix, respectively.

FIG. 10A-10B show that treatment of moDCs with R848 in combination with LNPs loaded with mRNA encoding cGASΔN leads to hyperactivation, which is defined by the ability of DCs to secrete IL-1β while remaining viable. moDCs were either left unstimulated (PBS treated), or treated with LNPs loaded with mRNA encoding cGASΔN or OVA in the presence of media, R848, or R848 and MCC950. FIG. 10A shows that all treatment conditions were conducive to viability, indicating that the cells were not pyroptosing in response to any of the treatment conditions. FIG. 10B shows that only treatment with LNPs loaded with mRNA encoding cGASΔN, when delivered in combination with R848, stimulated moDCs to secrete IL-1β. Treatment of moDCs with the selective NLRP3 inflammasome inhibitor MCC950 eliminated IL-1β production that would have otherwise been induced by treatment with the cGASΔN mRNA-loaded LNPs in combination with R848. P<0.0001****. Statistics were completed using two-way ANOVA with a Tukey post test for multiple comparisons.

FIG. 11A-11D show that administration of LNPs loaded with cGASΔN mRNA increases T cell responses to an mRNA-encoded model antigen delivered via LNP. Mice were immunized twice (prime and boost regimen) with LNPs loaded with 5 mcg mRNA encoding ovalbumin (OVA) delivered in combination with LNPs loaded with 1 mcg or 5 mcg cGASΔN mRNA or loaded with 1 mcg or 5 mcg green fluorescent protein (GFP) mRNA. Blood was collected 7 days post-boost and binding of “SIINFEKL-tetramers” to T cells was subsequently assessed. SIINFEKL is the amino acid sequence (SEQ ID NO:17) of a T cell epitope within OVA. Treatment of mice with LNPs loaded with cGASΔN mRNA in combination with LNPs loaded with mRNA-encoding OVA significantly increased both the frequency (FIG. 11A) and absolute number (FIG. 11C) of SIINFEKL-specific CD8+ T cells in the blood as compared to treatment with LNPs loaded with mRNA encoding OVA and GFP or mRNA encoding GFP and cGASΔN in LNPs prepared using the GenVoy ILM™ formulation. Similarly, treatment of mice with LNPs loaded with cGASΔN mRNA in combination with LNPs loaded with mRNA-encoding OVA significantly increased both the frequency (FIG. 11B) and absolute number (FIG. 11D) of SIINFEKL-specific CD8+ T cells in the blood as compared to treatment with LNPs loaded with mRNA encoding OVA and GFP in LNPs prepared using the custom lipid mix. P<0.01**, p<0.001***. Statistics for GenVoy ILM™ plots were completed using two-way ANOVA with a Tukey post test for multiple comparisons. Statistics for the custom lipid mix were completed using an unpaired t test.

FIG. 12A-12F show that treatment with LNPs loaded with cGASΔN mRNA reduces Th2 specific antibodies without impacting total IgG responses. Mice were immunized twice with LNPs loaded with 5 mcg OVA mRNA delivered with LNPs loaded with 1 mcg or 5 mcg cGASΔN mRNA or LNPs loaded with 1 mcg or 5 mcg of GFP mRNA. Blood was collected 7 days post-boost and serum was isolated to quantify antibody responses. Mice treated with LNPs loaded with OVA and cGASΔN mRNA did not show significant changes in OVA-specific total IgG as compared to mice treated with LNPs loaded with OVA mRNA and GFP mRNA in LNPs prepared using the GenVoy ILM™ formulation (FIG. 12A) and in LNPs prepared using the custom lipid mix (FIG. 12B). As shown in FIG. 12C, OVA-specific IgG1 was significantly reduced by the presence cGASΔN mRNA in LNPs prepared using GenVoy ILM™ formulation (p<0.05 for the 1:500 and 1:2500 dilution, 1 mcg and 5 mcg cGASΔN mRNA-containing LNPs versus GFP mRNA-containing LNPs. As shown in FIG. 12D, OVA-specific IgG1 responses were reduced in mice treated with cGASΔN mRNA-containing LNPs prepared using the custom lipid mix (p<0.05 for the 1:500, 1:2500, and 1:12500 dilution at 5 mcg cGASΔN mRNA-containing LNPs versus GFP mRNA-containing LNPs. In contrast, mice treated with OVA mRNA containing and cGASΔN LNPs did not show significant changes in OVA-specific IgG2a measured compared to OVA+GFP LNP treatment when LNPs were prepared with the GenVoy (E) and custom lipid mix (F) formulations. Legend at top applies to all panels. Statistics were completed using two-way ANOVA with a Tukey post test for multiple comparisons

FIG. 13A shows structures of cationic and ionizable lipids suitable for use in the lipid-based mRNA delivery vehicles of the present disclosure. FIG. 13B shows structures of other types of lipids suitable for use in the lipid-based mRNA delivery vehicles of the present disclosure. See also, Hou et al., Nature Review Materials, 6:1078-1094, 2021, which is incorporated herein by reference.

### DETAILED DESCRIPTION

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising a pathogen recognition receptor agonist. In some embodiments, the compositions of the present disclosure do not comprise a lysophosphatidylcholine (LPC) with a single C13-C24 acyl chain.

Inducing an inflammatory response can be desirable, such as for an immunotherapy or vaccination. COVID-19 mRNA vaccines (comprising LNPs loaded with mRNA encoding a SARS-CoV-2 antigen) have proven to be effective at reducing frequency and severity of infections. As described herein, inducing the cGAS-STING innate immune pathway adjuvants compositions comprising LNPs loaded with mRNA encoding a protein antigen, which in exemplary embodiments is ovalbumin (OVA). STING signaling is of particular interest out of all possible innate immune signaling pathways because, unlike some other signaling pathways, cGAS-STING activation does not induce translation inhibition.

In order to induce cGAS-STING signaling, a cGAS mutant lacking a portion of its N-terminus was designed such that it is constitutively active (cGASΔN). mRNA encoding the cGAS mutant was packaged in an LNP for cellular uptake and protein expression. The LNP-packaged mRNA encoding cGASΔN has the ability to adjuvant an immune response via the cGAS-STING pathway. Importantly, inclusion of mRNA encoding a constitutively active cGAS, such as cGASΔN, increases the potency of LNPs loaded with mRNA encoding an antigen by increasing inflammatory signals.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 810 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition comprising one or more mRNAs encoding an antigen and a constitutively-active cGAS, an effective amount contains sufficient mRNA, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). As an example, when used in reference to a phospholipid, an isolated phospholipid is at least 90%, 95%, 96%, 97%, 98% or 99% pure as determined by thin layer chromatography, or gas chromatography. As a further example, when used in reference to a recombinant protein, an isolated protein refers to a protein that has been removed from the culture medium of the host cell that produced the protein.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. In the context of the present disclosure, the term “antigen” typically refers to a polypeptide encoded by a nucleic acid sequence of a mRNA or a DNA. Polypeptide antigens are preferably at least eight amino acid residues in length, and may comprise one or more post-translational modifications.

The term “agonist” is used in the broadest sense and includes any molecule that activates signaling through a receptor. In some embodiments, the agonist binds to the receptor. For instance, a TLR8 agonist binds to a TLR8 and activates a TLR8-signaling pathway.

“Alkyl” refers to monovalent saturated aliphatic hydrocarbyl groups. Cx alkyl refers to an alkyl group having x number of carbon atoms. Cx-Cy alkyl or Cx-y alkyl refers to an alkyl group having between x number and y number of carbon atoms, inclusive.

“Alkylene” refers to divalent saturated aliphatic hydrocarbyl groups.

“Alkenyl” refers to monovalent hydrocarbyl groups having at least one double bond (>C═C<). Cx alkenyl refers to an alkenyl group having x number of carbon atoms. Cx-Cy alkenyl or Cx-y alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition of the present disclosure as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition that is at least 2, 3, 4, 5, 6, 7, 8, 9, or 10-fold above a level of DC hyperactivation as a consequence of a control condition. Likewise, a “lower level of DC hyperactivation” refers to a level of DC hyperactivation as a consequence of a treatment condition that is at least 2, 3, 4, 5, 6, 7, 8, 9, or 10-fold below a level of DC hyperactivation as a consequence of a control condition.

As used herein the term “immunization” refers to a process that increases a mammalian subject's immune response to an antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen or a nucleic acid encoding an antigen, enhances or potentiates an immune response to the antigen in the mammalian recipient upon exposure.

The terms “treating” or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

The term “constitutively-active” as used herein in reference to cGAS refers to a cGAS variant that binds to DNA in the cytoplasm and catalyzes cGAMP synthesis even under some conditions in which native cGAS has little to no enzymatic activity. In some preferred embodiments, the “constitutively-active cGAS” is a “truncated cGAS”, such as cGASΔN comprising a C-terminal DNA-binding, enzymatic domain in the absence of a N-terminal disordered domain. That is, cGASΔN is a constitutively-active cGAS devoid of regulation of enzymatic activity imparted by the N-terminal disordered domain of full length cGAS.

### I. Constitutively-Active Cyclic GMP-AMP Synthase

Cyclic GMP-AMP synthase, also referred to as cGAMP synthase or cGAS, is an enzymatic sensor of cytosolic DNA. cGAS recognizes double-stranded DNA independent of its sequence resulting in dimerization, formation of liquid-like droplets and production of the secondary messenger 2′3′cyclic GMP-AMP (cGAMP), which binds to and activates STING resulting in expression of interferons and other inflammatory mediators. Specifically, STING activation results in a signaling cascade that ultimately activates IRF3 and NF-kB transcription factors. IRF3 activation leads to the expression of genes such as IP-10 and Type I interferons that induce antiviral immune responses. NF-kB activation induces the expression of inflammatory cytokines such as IL-6 that enhance inflammatory immune responses.

Human cGAS is 522 amino acids in length, including a N-terminal phosphoinositide-binding domain (residues 1-59) and a C-terminal DNA-binding and enzymatic domain (residues 160-522) (Barnett et al., Cell, 176:1432-1446, 2019). Importantly, expression of cGASΔN in a human leukemia monocytic cell line was found to result in higher levels of expression of interferon and interferon-stimulated genes (Barnett, supra, 2019).

The amino acid sequence of human cGAS (GenBank No. NP_612450.2) is:

(SEQ ID NO: 10)
MQPWHGKAMQRASEAGATAPKASARNARGAPMDPTESPAAPEAALPKAGK
FGPARKSGSRQKKSAPDTQERPPVRATGARAKKAPQRAQDTQPSDATSAP
GAEGLEPPAAREPALSRAGSCRQRGARCSTKPRPPPGPWDVPSPGLPVSA
PILVRRDAAPGASKLRAVLEKLKLSRDDISTAAGMVKGVVDHLLLRLKCD
SAFRGVGLLNTGSYYEHVKISAPNEFDVMFKLEVPRIQLEEYSNTRAYYF
VKFKRNPKENPLSQFLEGEILSASKMLSKFRKIIKEEINDIKDTDVIMKR
KRGGSPAVTLLISEKISVDITLALESKSSWPASTQEGLRIQNWLSAKVRK
QLRLKPFYLVPKHAKEGNGFQEETWRLSFSHIEKEILNNHGKSKTCCENK
EEKCCRKDCLKLMKYLLEQLKERFKDKKHLDKFSSYHVKTAFFHVCTQNP
QDSQWDRKDLGLCFDNCVTYFLQCLRTEKLENYFIPEFNLFSSNLIDKRS
KEFLTKQIEYERNNEFPVEDEF.

The amino acid sequence of the N-terminal domain of cGAS is:

(SEQ ID NO: 11)
MQPWHGKAMQRASEAGATAPKASARNARGAPMDPTESPAAPEAALPKAGK
FGPARKSGSRQKKSAPDTQERPPVRATGARAKKAPQRAQDTQPSDATSAP
GAEGLEPPAAREPALSRAGSCRQRGARCSTKPRPPPGPWDVPSPGLPVSA
PILVRRDAA.

The amino acid sequence of the C-terminal domain of cGAS (cGASΔN) is:

(SEQ ID NO: 1)
PGASKLRAVLEKLKLSRDDISTAAGMVKGVVDHLLLRLKCDSAFRGVGLL
NTGSYYEHVKISAPNEFDVMFKLEVPRIQLEEYSNTRAYYFVKFKRNPKE
NPLSQFLEGEILSASKMLSKFRKIIKEEINDIKDTDVIMKRKRGGSPAVT
LLISEKISVDITLALESKSSWPASTQEGLRIQNWLSAKVRKQLRLKPFYL
VPKHAKEGNGFQEETWRLSFSHIEKEILNNHGKSKTCCENKEEKCCRKDC
LKLMKYLLEQLKERFKDKKHLDKFSSYHVKTAFFHVCTQNPQDSQWDRKD
LGLCFDNCVTYFLQCLRTEKLENYFIPEFNLFSSNLIDKRSKEFLTKQIE
YERNNEFPVFDEF.

The nucleotide sequence encoding human cGASΔN, which was codon-optimized for expression in mouse cells is set forth as SEQ ID NO:17. A DNA template with this nucleotide sequence was used to prepare mRNA encoding human cGASΔN, which was loaded into LNPs and tested as described in Examples 1, 2 and 3.

Compositions and methods of the present disclosure comprise a nucleic acid encoding a constitutively-active cGAS as a catalytic adjuvant for improving adaptive immune responses elicited by mRNA vaccines. In some preferred embodiments, the constitutively-active cGAS is a truncated cGAS devoid of the N-terminal phosphoinositide-binding domain (cGASΔN). In some preferred embodiments, the constitutively-active cGAS is a truncated cGAS comprising the C-terminal DNA-binding and enzymatic domain (cGASΔN).

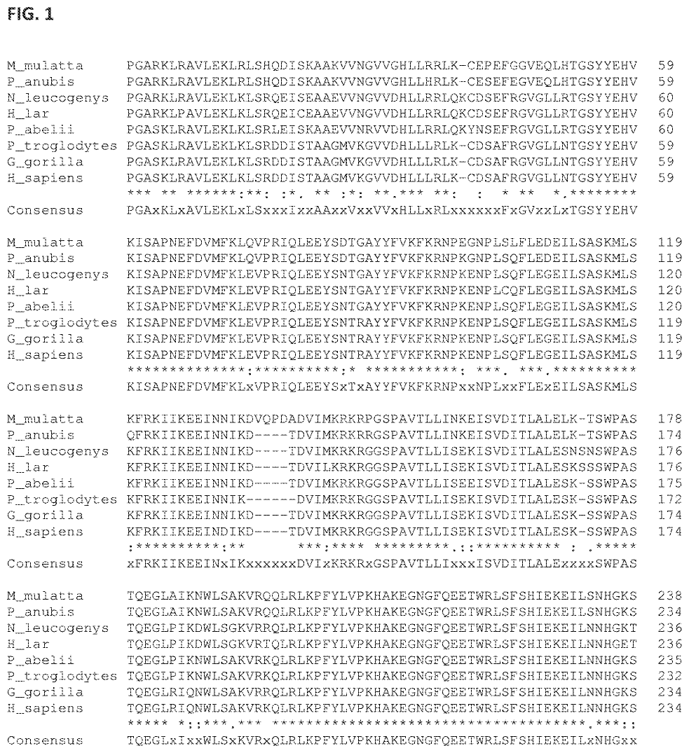

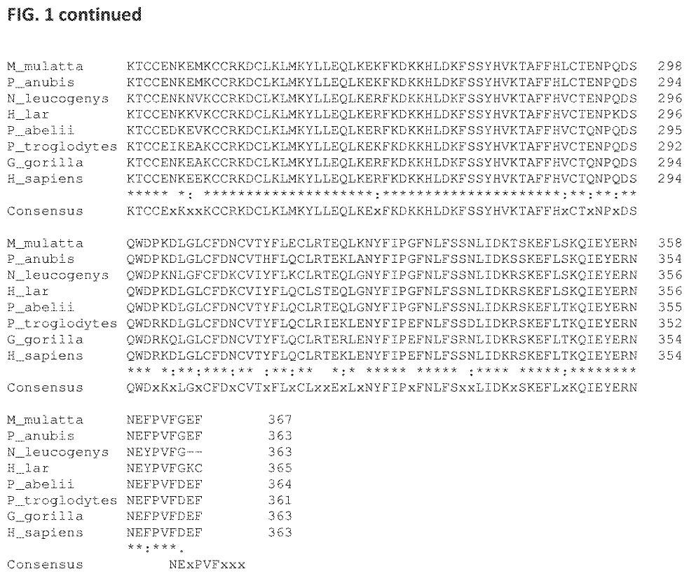

Homologs of cGAS are expressed in species across the animal kingdom, and cGAS amino acid sequences are conserved in higher primates. An alignment of the amino acid sequence of the c-terminal domain of human cGAS with the amino acid sequences of the c-terminal domains of multiple non-human primate cGAS proteins is shown in FIG. 1.

TABLE I
Percent Identity Matrix Created by Clustal 12.1\hat{ }

| # | Species | 1 | 2 | 3 | 4 | 5 | 6 | 7 | 8 |
|---|---|---|---|---|---|---|---|---|---|
| 1 | M._{—}mulatta | 100.00 | 96.42 | 87.53 | 85.95 | 88.71 | 87.26 | 86.78 | 86.50 |
| 2 | P._{—}anubis | 96.42 | 100.00 | 87.53 | 86.23 | 88.98 | 88.09 | 87.88 | 87.33 |
| 3 | N._{—}leucogenys | 87.53 | 87.53 | 100.00 | 96.14 | 93.37 | 89.97 | 90.03 | 90.30 |
| 4 | H_{—}lar | 85.95 | 86.23 | 96.14 | 100.00 | 92.31 | 88.92 | 88.98 | 88.71 |
| 5 | P._{—}abelii | 88.71 | 88.98 | 93.37 | 92.31 | 100.00 | 93.07 | 93.39 | 92.84 |
| 6 | P._troglodytes | 87.26 | 88.09 | 89.97 | 88.92 | 93.07 | 100.00 | 97.51 | 96.95 |
| 7 | H._{—}sapiens | 86.78 | 87.88 | 90.03 | 88.98 | 93.39 | 97.51 | 100.00 | 98.35 |
| 8 | G._{—}gorilla | 86.50 | 87.33 | 90.30 | 88.71 | 92.84 | 96.95 | 98.35 | 100.00 |

\hat{ }Numbers in this table are not SEQ ID NOS.

In some preferred embodiments, the constitutively-active cGAS is a truncated human cGAS devoid of the N-terminal domain (SEQ ID NO:11). In some preferred embodiments, the constitutively-active cGAS is a truncated human comprising the C-terminal domain (SEQ ID NO:1). In some preferred embodiments, cGASΔN comprises the amino acid sequence of SEQ ID NO:1 or the amino acid sequence that is at least 85%, 90%, 95%, 96%, 97%, 98% or 99% identical to SEQ ID NO:1. In some preferred embodiments, cGASΔN comprises the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8. In some preferred embodiments, cGASΔN comprises the consensus amino acid sequence of SEQ ID NO:9. For expression in transfected cells, the nucleic acid encoding cGASΔN is in operable combination with a start codon (ATG).

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows: 100 times the fraction X/Y, where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

II. mRNA Encoding an Antigen

Compositions and methods of the present disclosure may comprise an mRNA encoding an antigen or are otherwise suitable for use with a formulation comprising an mRNA encoding an antigen. In some embodiments, the antigen is a proteinaceous antigen. The terms “polypeptide” and “protein” are used interchangeably herein in reference to antigens that comprise peptide chains that are at least 8 amino acids in length. In some embodiments, the antigen is from 8 to 1800 amino acids, 9 to 1000 amino acids, or 10 to 100 amino acids in length. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe.

In some preferred embodiments, the mRNA comprises a 5′ untranslated region (5′UTR) at the 5′ end of the coding region and a 3′ untranslated region (3′UTR) at the 3′ end of the coding region. In some preferred embodiments, the mRNA comprises one or both of a 5′ cap structure and a polyA tail.

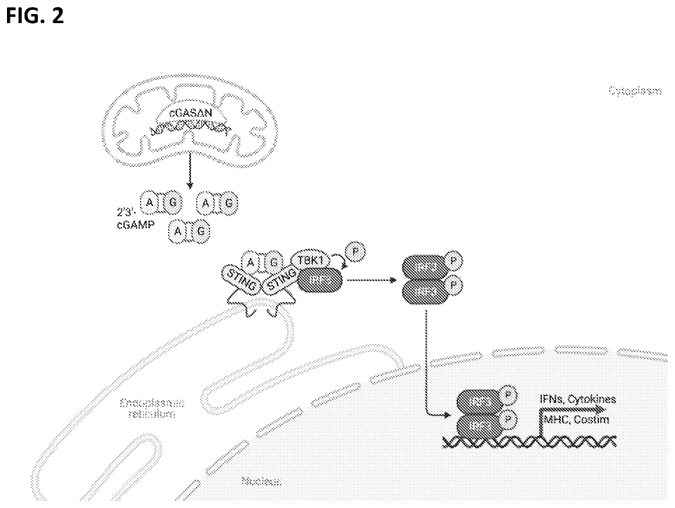

In some embodiments the mRNA further encodes a ribosome skipping sequence, such as the 2A-like sequence set forth as SEQ ID NO: 12. Additional 2AL sequences are set forth in SEQ ID NO:13, SEQ ID NO:14, and SEQ ID NO:15. In some embodiments in which the mRNA comprises two or more coding regions, the coding regions may be separated by a 2AL sequence. For instance, the 2AL sequence may be located between the coding region of constitutively active cyclic GMP-AMP synthase (cGAS), and the coding region of an antigen (e.g., cGASΔNs-2AL-antigen or antigen-2AL-cGASΔN). Similarly, in some embodiments in which the mRNA comprises coding regions for two or more antigens, the coding regions may be separated by a 2AL sequence (e.g., antigen1-2AL-antigen2). Additional 2AL sequence for use in mRNAs of the present disclosure are known in the art (see, e.g., Luke et al., J. Gen. Virol, 89:1036-1042, 2008, 2AL sequences of FIG. 2 are incorporated herein by reference).

### III. Lipid-Based Delivery Vehicles

Compositions and methods of the present disclosure may comprise a lipid-based delivery vehicle for an mRNA vaccine. In some embodiments, the vehicle is a lipid nanoparticle (LNP). In other embodiments, the vehicle is a lipid that forms a complex with the mRNA (RNA-Lipoplex).

In some embodiments, the LNP comprises at least one lipid selected from the group consisting of an ionizable lipid, a cationic lipid, a phospholipid, a pegylated lipid, a structural lipid, and mixtures thereof. In some embodiments, the at least one lipid comprises an ionizable lipid. In some embodiments, the at least one lipid comprises a cationic lipid. In some embodiments, the at least one lipid comprises a phospholipid. In some embodiments, the at least one lipid comprises a pegylated lipid. In some embodiments, the at least one lipid comprises a structural lipid. In some embodiments, the at least one lipid comprise an ionizable lipid, a phospholipid, a pegylated lipid, and a structural lipid.

In some embodiments, the lipid component of RNA-Lipoplex comprises one or more lipids. In some preferred embodiments, the one or more lipids comprise a first lipid and a second lipid, wherein the first lipid is distinct from the second lipid. In some embodiments, the first lipid is a cationic lipid and the second lipid is a neutral or anionic lipid.

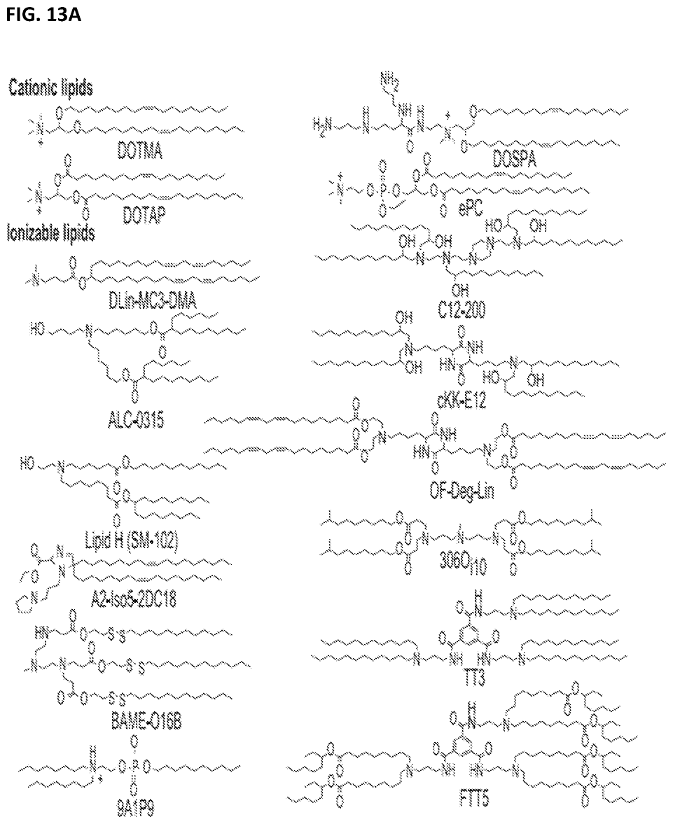

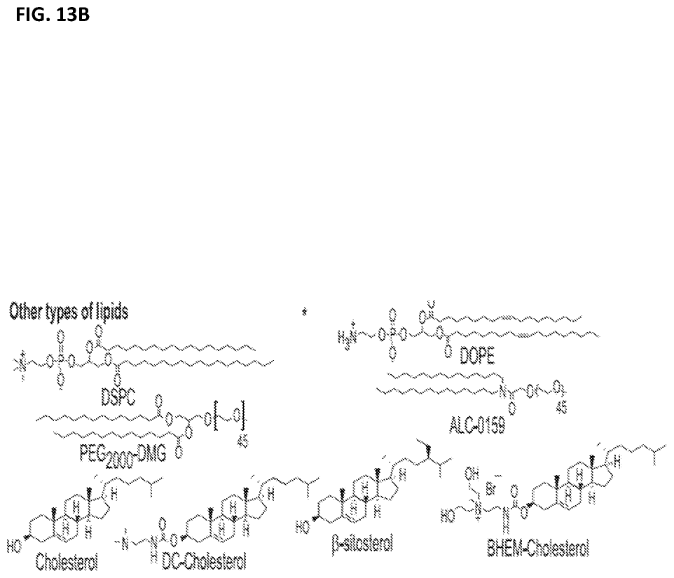

Structures of lipids suitable for use in the lipid-based mRNA delivery vehicles of the present disclosure are depicted in FIG. 13A and FIG. 13B, which are adapted from FIG. 2 of Hou et al., Nature Review Materials, 6:1078-1094, 2021.

### IV. Pathogen Recognition Receptor Agonists

Compositions and methods of the present disclosure may comprise a further pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In some embodiments, the PRR agonist comprises a TLR7/8 agonist.

### A. TLR7/8 Agonists

The term “TLR7/8 agonist” as used herein refers to an agonist of TLR7 and/or TLR8. In one aspect, the TLR7/8 agonist is a TLR7 agonist. In another aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of a DAMP such as oxPAPC or PGPC.

In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In other aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist.

### V. Pharmaceutical Formulations

Some compositions of the present disclosure are pharmaceutical formulations comprising a pharmaceutically acceptable excipient. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation.

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent).

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative.

The pharmaceutical formulations of the present disclosure are suitable for parenteral administration. That is the pharmaceutical formulations of the present disclosure are not intended for enteral administration (e.g., not by orally, gastrically, or rectally).

### VI. Methods of Use

In some aspects, the present disclosure relates to methods of use of any one of the compositions or formulations described herein. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating or preventing an infectious disease. The methods comprise administering an effective amount of a formulation or a composition described herein to an individual in need thereof to achieve a specific outcome. The individual is a mammalian subject, such as a human patient. In other embodiments, the individual a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), a or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” and immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or more of an antigen-specific antibody response, an antigen-specific cytotoxic T lymphocyte (CTL) response, and an antigen-specific helper T (Th) cell response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency. “Inducing” an antigen-specific Th cell response means increasing frequency of antigen-specific Th cells found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some preferred embodiments, at least 50%, 55%, 60%, 65%, 70% or 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease.

In some embodiments, the methods involve treating cancer in an individual or otherwise treating a mammalian subject with cancer. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### ENUMERATED EMBODIMENTS

In this section, Embodiment 1 refers to both Embodiment 1′ and Embodiment 1″
1′. A composition comprising a first mRNA encapsulated in a first lipid nanoparticle (LNP) and a second mRNA encapsulated in a second LNP,
wherein the first mRNA comprises a coding region of a constitutively active cyclic GMP-AMP synthase (cGAS),
wherein the second mRNA comprises a coding region of an antigen, and
wherein both the first LNP and the second LNP comprises a phospholipid, and at least one lipid selected from the group consisting of an ionizable lipid, a pegylated lipid, a structural lipid, and mixtures thereof.
1″. A composition comprising an mRNA encapsulated in a lipid nanoparticle (LNP), wherein the mRNA comprises a coding region of a constitutively active cyclic GMP-AMP synthase (cGAS), and the LNP comprises a phospholipid, and at least one lipid selected from the group consisting of an ionizable lipid, a pegylated lipid, a structural lipid, and mixtures thereof, optionally wherein the composition further comprises a further mRNA encapsulated in a further lipid nanoparticle, and the further mRNA comprises a coding region of an antigen of interest and the LNP comprises a phospholipid, and at least one lipid selected from the group consisting of an ionizable lipid, a pegylated lipid, a structural lipid, and mixtures thereof.
2. A composition comprising a first mRNA and a second mRNA encapsulated in a lipid nanoparticle (LNP), wherein the first mRNA comprises a coding region of a constitutively active cyclic GMP-AMP synthase (cGAS), the second mRNA comprising a coding region of an antigen; and the LNP comprises a phospholipid, and at least one lipid selected from the group consisting of an ionizable lipid, a pegylated lipid, a structural lipid, and mixtures thereof.
3. A composition comprising an mRNA encapsulated in a lipid nanoparticle (LNP), wherein the mRNA comprises a first coding region and a second coding region separated by a 2A-like sequence, wherein the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the LNP comprises a phospholipid, and at least one lipid selected from the group consisting of an ionizable lipid, a pegylated lipid, a structural lipid, and mixtures thereof.
4. The composition of any one of embodiments 1-3, wherein the at least one lipid comprises an ionizable lipid, a pegylated lipid, and a structural lipid.
5. The composition of any one of embodiments 1-4, wherein the ionizable lipid comprises:
i) 8-[(2-hydroxyethyl)[6-oxo-6-(undecyloxy)hexyl]amino]-octanoic acid, 1-octylnonyl ester (SM-102) or analogs or derivatives thereof; and/or
ii) 6-((2-hexyldecanoyl)oxy)-N-(6-((2-hexyldecanoyl)oxy)hexyl)-N-(4-hydroxybutyl)hexan-1-aminium (ALC-0315) or analogs or derivatives thereof, and/or
iii) (6Z,9Z,28Z,31Z)-heptatriaconta-6,9,28,31-tetraen-19-yl 4-(dimethylamino)butanoate (DLin-MC3-DMA)ALC-0315 or analogs or derivatives thereof.
6. The composition of any one of embodiments 1-5, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.
7. The composition of any one of embodiments 1-5, wherein the pegylated lipid comprises polyethylene glycol [PEG]2000 dimyristoyl glycerol [DMG].
8. The composition of any one of embodiments 1-7, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.
9. The composition of any one of embodiments 1-7, wherein the structural lipid comprises cholesterol.
10. The composition of any one of embodiments 1-9, wherein the phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.
11. The composition of any one of embodiments 1-9, wherein the phospholipid is selected from the group consisting of:
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanola mine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
   combinations thereof.
   12. The composition of embodiment 11, wherein the phospholipid comprises 1,2-distearoyl-sn-glycero-3-phosphocholine (DSPC).
   13. The composition of any one of embodiments 1-12, wherein the composition further comprises at least one excipient, optionally wherein the excipient comprises sucrose.
   14. The composition of any one of embodiments 1-13, wherein the composition does not comprise a lysophosphatidylcholine (LPC), optionally wherein the LPC has a single C13-C24 acyl chain.
   15. A composition comprising:
   an mRNA complexed with one or more lipids (RNA-Lipoplex); and
   wherein the mRNA comprises a coding region of a constitutively active cyclic GMP-AMP synthase (cGAS), and the one or more lipids comprise a first lipid and a second lipid.
   16. A composition comprising:
   a first mRNA and a second mRNA complexed with one or more lipids (RNA-Lipoplex); and
   wherein the first mRNA comprises a coding region of a constitutively active cyclic GMP-AMP synthase (cGAS), and the second mRNA comprises a coding region of an antigen, and the one or more lipids comprise a first lipid and a second lipid.
   17. A composition comprising:
   an mRNA complexed with one or more lipids (RNA-Lipoplex); and
   wherein the mRNA comprises a first coding region and a second coding region separated by a 2A-like sequence, the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the one or more lipids comprise a first lipid and a second lipid.
   18. The composition of any one of embodiments 15-17, wherein the first lipid is a cationic lipid, and the second lipid is a neutral or anionic lipid.
   19. The composition of embodiment 18, wherein the cationic lipid comprises one or both of:
   i) 1,2-di-O-octadecenyl-3-trimethylammonium propane (DOTMA) or analogs or derivatives thereof; and
   ii) 1,2-dioleoyl-3-trimethylammonium propane (DOTAP) or analogs or derivatives thereof.
   20. The composition of embodiment 18 or embodiment 19, wherein the neutral or anionic lipid comprises:
   i) 1,2-di-(9Z-octadecenoyl)-sn-glycero-3-phosphoethanolamine (DOPE) or analogs or derivatives thereof; and/or
   ii) cholesterol or analogs or derivatives thereof, and/or
   iii) 1,2-dioleoyl-sn-glycero-3-phosphocholine (DOPC) or analogs or derivatives thereof.
   21. The composition of any one of embodiments 1-20, further comprising a TLR7/8 agonist.
   22. The composition of embodiment 21, wherein the TLR7/8 agonist is a small molecule with a molecule weight of 900 daltons or less.
   23. The composition of embodiment 22, wherein the TLR7/8 agonist comprises an imidazoquinoline compound.
   24. The composition of embodiment 23, wherein the TLR7/8 agonist comprises resiquimod (R848).
   25. The composition of any one of embodiments 1-24, wherein the mRNA or the first mRNA and the second mRNA comprises a 5′ untranslated region (5′UTR) and a 3′ untranslated region (3′UTR).
   26. The composition of any one of embodiments 1-25, wherein the mRNA comprises a 5′ cap structure.
   27. The composition of any one of embodiments 1-26, wherein the mRNA comprises a polyA tail.
   28. The composition of any one of embodiments 1-27, wherein the mRNA is a nucleoside-modified mRNA.
   29. The composition of any one of embodiments 1-28, wherein the constitutively-active cGAS has a greater propensity to self DNA reactivity than its wild-type counterpart.
   30. The composition of any one of embodiments 1-29, wherein the cGAS is a truncated cGAS devoid of an amino-terminal phosphoinositide-binding domain (cGASΔN).
   31. The composition of embodiment 30, wherein the cGASΔN comprises the amino acid sequence of SEQ ID NO:1 or the amino acid sequence at least 85%, 90%, 91%, 92%, 93%, 94%, 95%, 96%, 97%, 98%, or 99% identical to SEQ ID NO:1.
   32. The composition of embodiment 31, wherein the cGASΔN comprises:
   (i) the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8; or
   (ii) the consensus amino acid sequence of SEQ ID NO:9.
   33. The composition of embodiment 31, wherein the cGASΔN is encoded by the nucleotide sequence of SEQ ID NO:17.
   34. The composition of any one of embodiments 30-33, wherein the coding region of the cGASΔN is in operable combination with a start codon (ATG).
   35. The composition of any one of embodiments 1-34, wherein the antigen is a tumor antigen.
   36. The composition of embodiment 35, wherein the tumor antigen is a tumor-associated antigen.
   37. The composition of embodiment 35, wherein the tumor antigen is a neoantigen.
   38. The composition of any one of embodiments 1-34, wherein the antigen comprises a microbial antigen.
   39. The composition of embodiment 38, wherein the microbial antigen comprises a viral antigen, a bacterial antigen, a protozoan antigen, or a fungal antigen.
   40. The composition of any one of embodiments 1-34, wherein the antigen comprises a surface protein or fragment thereof of a pathogen.
   41. The composition of embodiment 40, wherein the pathogen is capable of causing disease in human subjects.
   42. The composition of embodiment 40 or embodiment 41, wherein the pathogen is a virus.
   43. The composition of embodiment 42, wherein the virus is a SARS-CoV-2.
   44. The composition of embodiment 43, wherein the antigen is a spike (S) glycoprotein of the SARS-CoV-2, optionally wherein the spike glycoprotein is a pre-fusion stabilized variant.
   45. The composition of any one of embodiments 1-44, wherein the composition does not comprise lipopolysaccharide (LPS) or monophosphoryl lipid A (MPLA).
   46. The composition of any one of embodiments 1-45, wherein the composition does not comprise oxidized 1-palmitoyl-2-arachidonoyl-sn-glycero-3-phosphorylcholine (oxPAPC) or a species of oxPAPC.
   47. The composition of embodiment 46, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).
   48. A pharmaceutical formulation comprising the composition of any one of embodiments 1-47, and a pharmaceutically acceptable excipient.
   49. A method for production of hyperactivated dendritic cells, the method comprising contacting the dendritic cells with an effective amount of the composition of any one of embodiments 1-47, any one of embodiments 21-47, or the formulation of embodiment 48 to produce hyperactivated dendritic cells, wherein the hyperactivated dendritic cells secrete IL-1beta without undergoing cell death within about 48 hours of exposure.
   50. The method of embodiment 49, wherein the dendritic cells are:
   (i) contacted in vivo with the composition; or
   (ii) contacted ex vivo with the composition.
   51. The method of embodiment 49 or embodiment 50, wherein the hyperactivated dendritic cells:
   (i) secrete higher levels of one or more of RANTES, IP-10 and IFNα than unstimulated dendritic cells or dendritic cells contacted with empty LNPs; and/or
   (ii) express higher levels of at least one cell surface marker selected from the group consisting of CD40, CD86, CD69, MHC class II, MHC class I, CCR7, and combinations thereof.
   52. A pharmaceutical formulation comprising at least 10\hat{ }3, 10\hat{ }4, 10\hat{ }5 or 10\hat{ }6 of the hyperactivated dendritic cells produced by the method of any one of embodiments 49-51, and a pharmaceutically acceptable excipient.
   53. A method of stimulating an immune response against an antigen, comprising administering an effective amount of the pharmaceutical formulation of embodiment 48 or embodiment 52 to an individual in need thereof to stimulate the immune response against the antigen.
   54. A method of treating cancer, comprising administering an effective amount of the pharmaceutical formulation of embodiment 48 or embodiment 52 to an individual in need thereof to treat the cancer.
   55. A method of inhibiting abnormal cell proliferation, comprising administering an effective amount of the pharmaceutical formulation of embodiment 48 or embodiment 52 to an individual in need thereof to inhibit abnormal cell proliferation.
   56. A method of treating or preventing an infectious disease, comprising administering an effective amount of the pharmaceutical formulation of embodiment 48 to an individual in need thereof to treat or prevent the infectious disease.
   57. The method of embodiment 56, wherein the infectious disease is a viral disease.
   58. The method of embodiment 56, wherein the infectious disease is a bacterial disease.
   59. The method or pharmaceutical formulation of any one of embodiments 49-55, wherein the dendritic cells are mammalian cells.
   60. The method or pharmaceutical formulation of embodiment 59, wherein the mammalian cells are human cells.
   61. The method of any one of embodiments 53-59, wherein the individual is mammal.
   62. The method of embodiment 61, wherein the mammal is a human.
   63. The method of embodiment 61, wherein the mammal is a dog or a cat.
   64. The composition, formulation, or method of any one of embodiments 1-63, wherein the phospholipid comprises 1,2-distearoyl-sn-glycero-3-phosphocholine (DSPC), and the least one lipid comprises 8-[(2-hydroxyethyl)[6-oxo-6-(undecyloxy)hexyl]amino]-octanoic acid, 1-octylnonyl ester (SM-102) or analogs or derivatives thereof, and cholesterol.
   65. The composition, formulation, or method of embodiment 64, wherein the at least one lipid further comprises a pegylated lipid, optionally wherein the pegylated lipid comprises polyethylene glycol [PEG]2000 dimyristoyl glycerol [DMG].
   66. The composition, formulation, or method of any one of embodiments 1-65, wherein the LNP has an effective diameter of less than about 250 nanometers, optionally from about 25 to about 250 nanometers, optionally from about 50 to about 200 nanometers, or optionally from about 75 to about 175 nanometers.
   67. The composition, formulation, or method of embodiment 66, wherein the LNP has an effective diameter of less than about 200 nanometers.
   68. The composition, formulation, or method of claim 67, wherein the LNP has an effective diameter of less than about 150 nanometers.

### EXAMPLES

Abbreviations: BMDC (bone marrow-derived dendritic cell); CDS (cytosolic DNA sensor); cyclic GMP-AMP synthase (cGAS); CLR (C-type lectin receptor); DAMP (damage-associated molecular pattern); DC (dendritic cell); dLN (draining lymph node); DLS (dynamic light scattering); DMG-PEG-2000 (polyethylene glycol [PEG]2000 dimyristoyl glycerol [DMG]; DSPC (1,2-distearoyl-sn-glycero-3-phosphocholine); ELSD (evaporative light scattering detector); FLT3L (Fms-related tyrosine kinase 3 ligand); GFP (green fluorescent protein); GV (GenVoy ILM™ formulation); (HOdiA-PC (1-palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(palmitoyl)-2-(5-keto-6-octene-dioyl) phosphatidylcholine); KOOA-PC (1-palmitoyl-(5-keto-8-oxo-6-octenoyl)-sn-glycero-3-phosphocholine); LNP (lipid nanoparticle); LPC/Lyso PC (lysophosphatidylcholine); Lyso PC(22:0) (1-behenoyl-2-hydroxy-sn-glycero-3-phosphocholine); LPS (lipopolysaccharide); MC3 ((6Z,9Z,28Z,31Z)-heptatriaconta-6,9,28,31-tetraen-19-yl 4-(dimethylamino)butanoate, also referred to as DLin-MC3-DMA); mcg or μg (microgram); moDC (monocyte-derived dendritic cell); MPLA (monophosphoryl lipid A); NLR (NOD-like receptor); OVA (ovalbumin); oxPAPC (oxidized 1-palmitoyl-2-arachidonyl-sn-glycero-3-phosphorylcholine); PAMP (pathogen-associated molecular pattern); PBMCs (peripheral blood mononuclear cells); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); SC (subcutaneously); STING (stimulator of interferon genes); TNFα (tumor necrosis factor-alpha); and TLR (toll-like receptor).

Although the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1: Murine DCs Are Activatable In Vitro By LNPs Containing cGASΔN mRNA

### Materials & Methods

Materials. GenVoy ILM™ ionizable lipid mixture was purchased from Precision Nanosystems. CleanCap OVA and GFP mRNAs were purchased off-the-shelf from Trilink, with a 120 residue polyA tail, a 5-methoxyuridine base modification, and codon optimization for expression in mammalian systems. cGASΔN mRNA was custom ordered from Trilink and was synthesized via in vitro transcription from linearized template DNA. The nucleotide sequence of human cGASΔN sequence was codon optimized for expression in murine cells, and the synthetic nucleotide sequence is set forth as SEQ ID NO:17. The mRNA sequence was capped using Trilink's proprietary Clean Cap mRNA technology, with a N1-methylpseudouridine base modification, and a 120 residue polyA tail. The sequence contains a Bbsl restriction enzyme site. The mRNA was phosphatase treated after synthesis.

LNP Synthesis. Lipid nanoparticles (LNPs) were prepared using the GenVoy ILM™ ionizable lipid mixture (Precision Nanosytems). OVA mRNA, GFP mRNA, and cGASΔN mRNA (Trilink), were each prepared at 0.17 mg/mL in sodium citrate buffer, pH 4. GenVoy ILM™ was used at 12.5 mM. LNPs were synthesized using the NanoAssemblr Ignite instrument (Precision Nanosystems). Lipids in ethanol were combined with the mRNA solutions individually at a 1:3 volumetric ratio, using a flow rate of 12 mL/min. LNPs were washed in 10 volumes of phosphate buffered saline (PBS), pH 7.4 to remove residual ethanol, and then concentrated using Amicon 10K MWCO centrifugal filters. LNPs were filtered through a 0.2 μm filter before use.

LNP Characterization. Loading of mRNA into LNPs was quantified using a RiboGreen assay (ThermoFisher) following the manufacturer's protocol. Samples were diluted to fall within the range of the standard curve. LNPs were lysed using Triton X-100 to assess encapsulation of mRNA into LNPs. Both total mRNA and encapsulated mRNA were quantified. The size of the LNPs was assessed using dynamic light scattering (DLS) on the NanoBrook Omni (Brookhaven). LNPs were diluted 1:10 in PBS before running on the DLS. Three 90 second measurements were recorded for each sample.

Murine bone marrow-derived FLT3L-DCs generation. Leg femur and tibia were removed from mice, cut with scissors, and flushed into sterile tubes. Bone marrow suspension was treated with ACK Lysis Buffer for 1 minute, then passed through a 40 μm cell strainer. Cells were counted and resuspended in media consisting of complete IMDM containing 10% FBS, penicillin and streptomycin, and supplements of L-glutamine and sodium pyruvate (I10). Cells were then plated at 8×10⁶ bone marrow cells per well in a P12 plate. Recombinant mouse FLT3L (Miltenyi) was added to cultures at 200 ng/mL. Differentiated cells were used for subsequent assays on day 8. The efficiency of differentiation was monitored by flow cytometry using a BD Symphony A3, and CD11c⁺MHC-II⁺ cells were routinely above 80% of living cells. For each experiment, 5 to 15 mice were used to generate DCs from bone marrow.

Murine FLT3L-DCs stimulation. FLT3L-DCs were harvested 8 days post differentiation, counted and the treated with lipid nanoparticles (LNPs) based on the mRNA content. Cells were treated with 1 μg/mL mRNA in 200 μl total media/well. About 24 hours post incubation, cells and culture supernatant were used for downstream readouts. 150 mL of cell supernatant from each well was collected to measure cytokine secretion from DC. LEGENDplex™ Mouse Anti-Virus Response Panel (Biolegend) was used according to the manufacturer's protocol. Data were collected using a BD FACS Symphony and analyzed using the cloud-based software provided by Biolegend. To assess DC activation post LNP exposure, FLT3L DCs were collected 24 hours after LNP addition and stained to measure the expression of the following cell surface markers: CD11c, MHC-II, CD24, SIRP1a, CD40, CD86, CD69, and H2kb (MHC-I). Graphs show means and SD of triplicates. Data are representative of two experiments.

### Results

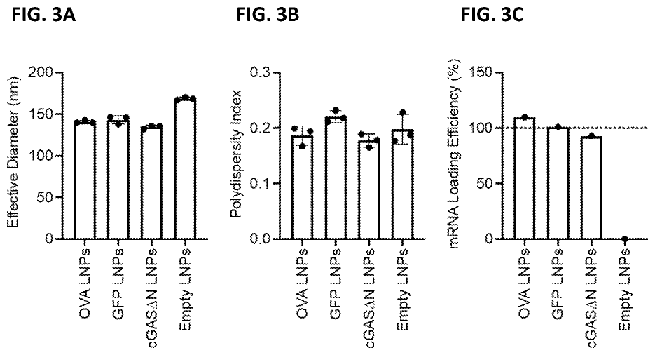

LNPs loaded with mRNA encoding cGASΔN (cGASΔNLNPs) exhibit similar sizing and loading profiles to LNPs loaded with mRNA encoding model antigens. cGASΔN mRNA loaded LNPs showed similar size and mRNA loading profiles to the LNPs loaded with model antigen mRNAs—OVA and GFP. All mRNA-loaded LNPs had an average effective diameter less than 150 nm (FIG. 3A), with a relatively uniform size profile, exhibited by polydispersity indexes less than 0.3 (FIG. 3B). All mRNAs loaded into the GenVoy ILM LNPs with the measured mRNA loading showing 90-110% of the theoretically loaded amount of mRNA (FIG. 3C).

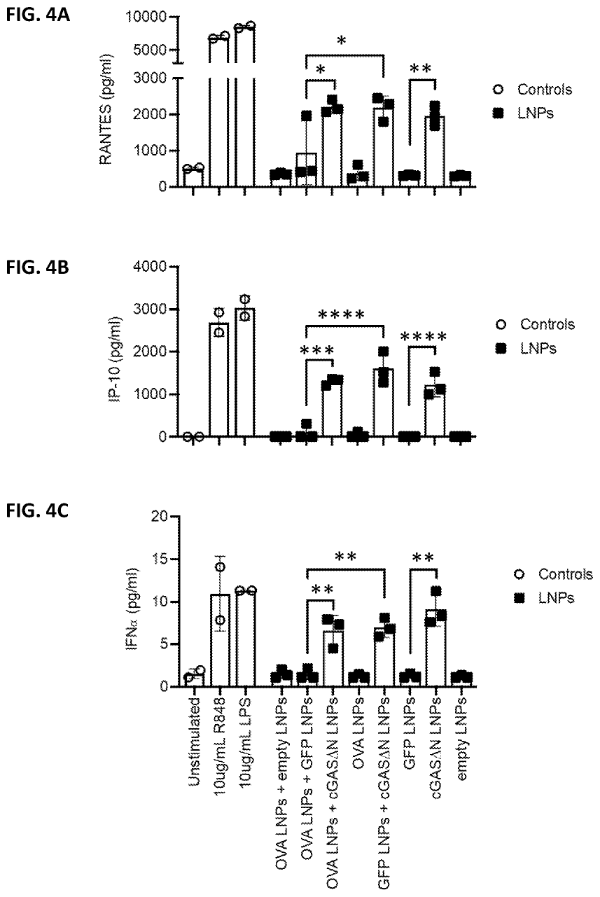

cGASΔNLNPs induce IFN-I-dependent response in murine DCs. FLT3L DCs were stimulated with empty LNPs (no mRNA), or LNPs containing either GFP mRNA or OVA mRNA or cGASΔN mRNA alone. Alternatively, DCs were stimulated with LNPs containing OVA mRNA plus empty LNPs or OVA mRNA plus LNPs containing cGASΔN or LNPs containing cGASΔN plus LNPs containing GFP mRNA. LNPs were added to cell cultures based on the concentration of mRNA at 1 μg/mL. 24 hours post stimulation, cell supernatants were measured for the presence of cytokines using a multiplexed cytokine bead array Legendplex assay. Interestingly, we found that upon cGASΔN LNP treatment, BMDCs secreted RANTES, IP-10, and IFNα at significantly higher levels than when treated with mRNA antigen-loaded LNPs alone (FIG. 4A, FIG. 4B, FIG. 4C). These data indicated that constitutively active cGASΔN is successfully expressed and is functional in mouse DCs. Pro-inflammatory cytokines dependent on NFkB were not secreted, including TL-6 and TNFα, suggesting that constitutively active cGASΔN induce NFkB activation in specific cell types. We compared the induction of these cytokine upon LNPs treatment to stimulation with LPS and R848 PAMPs. Interestingly IP-10, RANTES, and IFNα were expressed either more strongly or at similar levels by cGASΔN LNP treatment as compared to higher doses of LPS or R848 suggesting that the IRF3 pathway can be strongly induced by the expression of constitutively active cGASΔN.

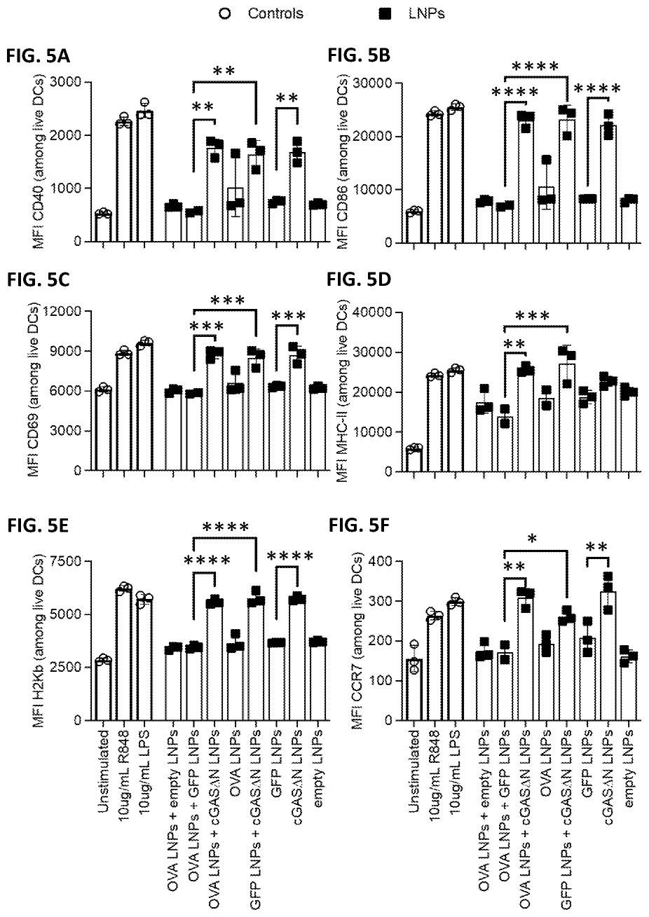

cGASΔNLNPs induce murine DCs activation. To assess DC activation, LNP-treated DCs were stained 24 hours following treatment with activation markers on total cDC as well as cDC1 and cDC2 subsets of DCs. The expression of T cell costimulatory surface molecules CD40, CD86 and CD69 increased when cells were treated with cGASΔN LNP but not with empty LNPs or GFP LNPs or OVA containing LNPs (FIG. 5A, FIG. 5B, FIG. 5C) when assessed by median fluorescence intensity (MFI). This trend also held true for cDC1 subsets. In addition, MHCII and H2kB antigen presentation molecules were increased on cDCs when they were treated with cGASΔN LNPs compared to standard mRNA antigen LNPs (FIG. 5D, 5E). This also held true for cDC1 subsets. Further, CCR7 surface expression, indicative of the potential for DCs to migrate to LN, was increased on cDCs when they were treated with cGASΔN LNPs compared to standard mRNA antigen LNPs (FIG. 5F). This trend was also true for cDC1 subsets. Overall, these data indicate that cGASΔN LNPs induce cGAS-STING pathway activation within DCs leading to a DC activation state and production of type-I IFN.

### Example 2: Human Cells are Activatable into an Inflammatory State in Vitro by LNPs Containing cGASΔN mRNA

### Materials & Methods

Materials. Lipids for LNPs were purchased from Cayman Chemicals (SM102) or Avanti (22:0 LPC, DSPC, DMG-PEG2000). Cholesterol was purchased from Sigma. GenVoy ILM lipids were purchased from Precision Nanosystems. CleanCap OVA and GFP mRNA were purchased off-the-shelf from Trilink, with a 120 polyA tail and base modification 5-methoxyuridine, optimized for mammalian systems. cGASΔN mRNA was custom ordered and synthesized via in vitro transcription from a linearized template DNA (Trilink). The human cGASΔN sequence was codon optimized to be expressed in mouse. The sequence was capped using Trilink's proprietary Clean Cap mRNA technology, with base modification N1-methylpseudouridine and a 120 polyA tail. The sequence contains a Bbsl restriction enzyme site. The sequence information was used to build plasmids, from which the mRNA was synthetically synthesized. mRNA was phosphatase treated after synthesis.

LNP Synthesis. Lipid nanoparticles (LNPs) were prepared using the GenVoy ILM LNP lipid mix (Precision Nanosytems) or a custom LNP lipid mix (Table 2-1). OVA mRNA, GFP mRNA, and cGASΔN mRNA (Trilink), were each prepared at 0.17 mg/mL in sodium citrate buffer, pH 4. GenVoy ILM and the custom LNP lipid mix were used at 12.5 mM. LNPs were synthesized using the NanoAssemblr Ignite instrument (Precision Nanosystems). Lipids in ethanol were combined with the mRNA solutions individually at a 1:3 volumetric ratio, using a flow rate of 12 mL/min. LNPs were washed in 10 volumes of phosphate buffered saline (PBS), pH 7.4 to remove residual ethanol, and then concentrated using Amicon 10K MWCO centrifugal filters. LNPs were filtered through a 0.2 μm filter before use.

TABLE 2-1
LNP Formulations

| Lipid | GenVoy ILM ™ | Custom Lipid Mix |
|---|---|---|
| Ionizable Lipid | Proprietary Lipid 50% | SM102 40% |
| DSPC | 10% | 35% |
| Cholesterol | 37.5% | 28.5% |
| Stabilizer Lipid | Proprietary Lipid 2.5% | DMG-PEG2000 1.5% |

LNP Characterization. Loading of mRNA into LNPs was quantified using a RiboGreen assay (ThermoFisher) following the manufacturer's protocol. Samples were diluted to fall within the range of the standard curve. LNPs were lysed using Triton X-100 to assess encapsulation of mRNA into LNPs. Both total mRNA and encapsulated mRNA were quantified. The size of the LNPs was assessed using dynamic light scattering (DLS) on the NanoBrook Omni (Brookhaven). LNPs were diluted 1:10 in PBS before running on the DLS. Three 90 second measurements were recorded for each sample.

Human monocyte derived dendritic cells (moDC) generation. Human monocytes were isolated from Leukopaks purchased from Miltenyi using the StraightFrom Leukopak CD14 microbead kit (Miltenyi). Isolations were completed following manufacturer's instructions. Monocytes were then aliquoted and frozen in fetal bovine serum containing 10% dimethyl sulfoxide. For studies with monocyte-derived dendritic cell (moDC) cultures, monocytes were thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (RIO media). To differentiate monocytes into moDCs, recombinant human GM-CSF (50 ng/mL) and IL-4 (25 ng/mL) were added to RIO media. Cells were cultured for 6 days with GM-CSF and IL-4, with an additional cell feeding with RIO media containing GM-CSF and IL-4 on day 3. Six days after differentiation, moDC were collected and counted. Cells were plated into 96-well flat-bottom plates at 1E5 cells/well in RIO media.

THP-1 cell generation. THP1-Null2 (Invivogen) cells were thawed into and maintained in RPMI medium containing 10% heat-inactivated FBS, 25 mM HEPES, 100 U/mL penicillin/streptomycin, 100 μg/mL normocin, and supplemented with 100 μg/mL zeocin every other passage. Cells were passaged between 0.4-0.6×10⁶ cells/mL in T75 culture flasks, with two cell passages per week. Cells are maintained until passage 20, after which a new vial was thawed. Before treating with LNPs, THP1-Null2 cells were collected from flasks and plated in RPMI medium containing 10% heat-inactivated FBS, 25 mM HEPES, and 100 U/mL penicillin/streptomycin at 100,000 cells/well in 96-well flat-bottom tissue culture plates.

Human cell activations. THP-1 cells and moDC were treated with lipid nanoparticles (LNPs) based on the concentration of mRNA content. Cells were treated with 0.2 μg/mL mRNA delivered in LNPs in a total stimuli volume of 200 μL/well. After an overnight incubation, cells and culture supernatant were used for downstream readouts. One hundred and fifty microliters of cell supernatant were collected. One day after LNP addition, cells and supernatant from THP-1 cultures were collected to measure IL-6 (Lumit assay by Promega) and IP-10 (ELISA kit from Biolegend) cytokines. THP-1 cells were used for staining activation markers for flow cytometry analysis. To measure cytokine secretion from moDC, the LegendPlex Anti-virus Response Panel (Biolegend) was used according to the manufacturer's protocol. Data were collected using a BD FACS Symphony and analyzed using the cloud-based software provided by Biolegend. Both moDC and THP-1 cells were collected 1 day after LNP addition and stained to measure the expression of the following cell surface activation markers: CD11c, CD209, CD40, CD80, CD83, CD86, HLA-ABC, and HLA-DR. Study was performed on two different human donor samples and on THP-1 cells, and each biological condition was tested in triplicate. Graphs show data from two human samples or THP-1 cells. Data is representative of two experiments.

HEK-Blue IL-1β Reporter Assay. moDC were treated with lipid nanoparticles (LNPs) based on the concentration of mRNA content. Cells were treated with 1 μg/mL OVA mRNA, 1 μg/mL cGASΔN mRNA, or 0.2 μg/mL mRNA delivered in LNPs in a total stimuli volume of 200 μL/well. LNPs were delivered with media, R848, or R848+MCC950, an inhibitor of NLRP3 inflammasomes. In the presence of MCC950, IL-1β should not be secreted in an inflammasome dependent manner. HEK-Blue IL-1β Reporter cells (Invivogen) were used to assess functional IL-1β secreted by human moDCs in response to treatment with cGASΔN LNPs for 48 hours. HEK-Blue IL-1β Reporter cells are derived from HEK293 cells, and express an NK-kB/AP-1 inducible SEAP (secreted embryonic alkaline phosphatase) reporter that triggers SEAP production once the IL-1β receptor encounters mature IL-1β. SEAP production is monitored by a color change using the QUANTI-Blue solution. HEK-Blue IL-1β Reporter cells were thawed into and maintained in DMEM medium containing 10% heat-inactivated FBS, 2 mM L-glutamine, 100 U/mL penicillin, 100 μg/mL streptomycin, 100 μg/mL normocin, and supplemented with 100 μg/mL zeocin every other passage. Cells were passaged between 0.4-0.6×10\hat{ }6 cells/mL in T75 culture flasks, with 2 cell passages per week. Cells are maintained until passage 20, after which a new vial is thawed. Before treating with LNPs, HEK-Blue IL-13 Reporter cells were collected from flasks and plated in DMEM medium containing 10% heat-inactivated FBS, 2 mM L-glutamine, 100 U/mL penicillin, and 100 μg/mL streptomycin at 50,000 cells/well in 96-well flat-bottom tissue culture plates. 50 μL of media from cGASΔN activated moDCs, or 50 μL of media containing known concentrations of recombinant human IL-1β to make a standard curve were added to wells containing HEK-Blue IL-1β Reporter cells. Cells were incubated overnight, after which 20 μL of SEAP-containing supernatant from incubated samples was mixed with 180 μL of QUANTI-Blue solution. This reaction ran for 1 hour at 37 C, after which absorbance at 630 nm was measured by spectrophotometry.

### Results

To determine if cGASΔN LNPs would allow for greater cell activation than standard LNPs, LNPs were synthesized to contain mRNA encoding Ova antigen or mRNA encoding constitutively active cGASΔN. As experimental controls, LNPs were made with mRNA encoding GFP. These LNPs were tested individually or in combination on THP-1 cells (a human myeloid cell line) and primary human moDC. Two lipid formulations: GenVoy (Precision Nanosystems) and a custom lipid mix were included to determine if activity was LNP-formulation dependent. LNPs were added to cell cultures based on the concentration of mRNA at 0.2 μg/mL.

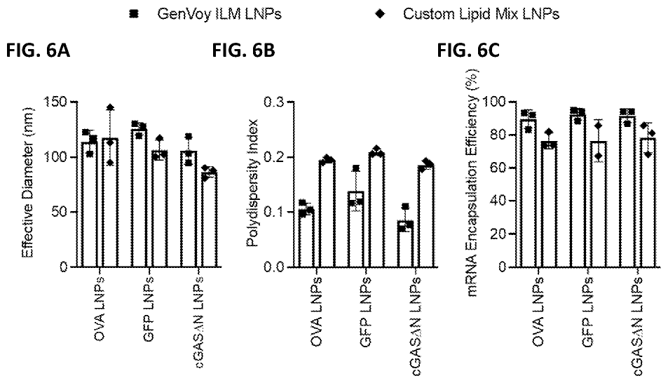

cGASΔNLNPs exhibit similar sizing and loading profiles to LNPs loaded with model antigens. cGASΔN mRNA loaded LNPs showed similar size and mRNA loading profiles to the LNPs loaded with model antigen mRNAs—OVA and GFP. All mRNA-loaded LNPs had an average effective diameter less than 150 nm (FIG. 6A), with a relatively uniform size profile, exhibited by polydispersity indexes less than 0.3 (FIG. 6B). All mRNAs loaded into the GenVoy ILM LNPs with 70-90% of the theoretically loaded amount of mRNA encapsulated in the LNPs (FIG. 6C). Both the GenVoy and the custom lipid mix allowed for LNP production, with similar sizing, although the optimized GenVoy formulation exhibited increased uniformity of size (FIG. 6B) as shown by a lower polydispersity index. Both the GenVoy and the custom lipid mix allowed for similar loading levels for of all mRNAs, with the GenVoy formulation encapsulating the mRNA slightly more efficiently.

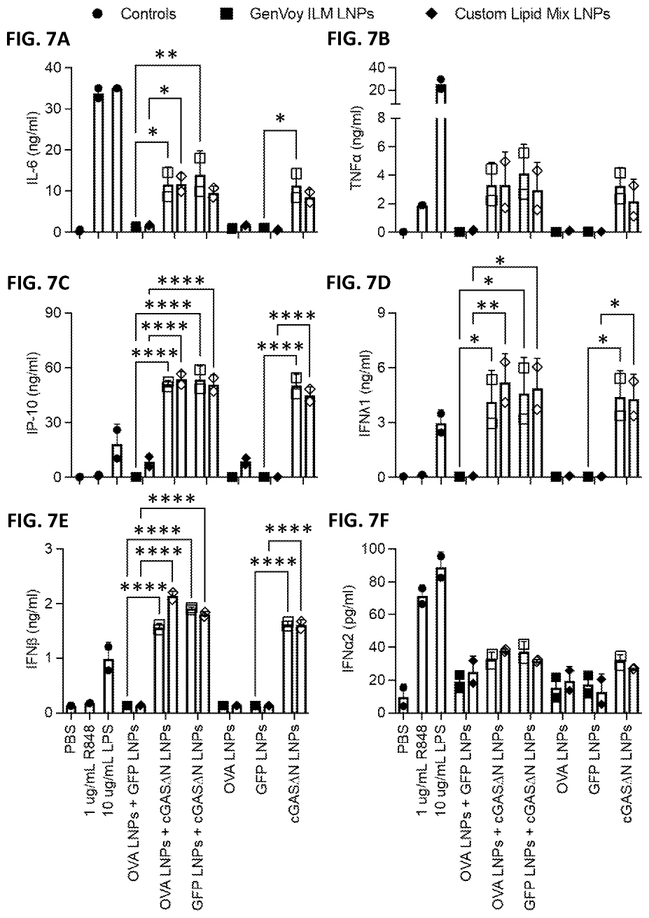

cGASΔNLNPs activate inflammatory pathways in human moDC. Frozen monocytes from two donors were thawed and differentiated into moDC using GM-CSF and IL-4. The cells were treated with LNPs at an mRNA loading of 0.2 μg/mL. Using a multiplexed cytokine bead array assay, multiple inflammatory cytokines were found to be upregulated. Upon cGASΔN LNP treatment, IL-6 (FIG. 7A), TNFα (FIG. 7B), IP-10 (FIG. 7C), IFN λ1 (FIG. 7D), IFNβ (FIG. 7E), and IFNα2 (FIG. 7F) were detectable at an elevated level within one day compared to treatment with LNPs loaded with model antigen. The expression of IL-6 suggested that constitutively active cGAS can induce NF-kB activation and is maybe cell type dependent. Interestingly IP-10, IFNλ1, and IFNβ were expressed more strongly by cGASΔN LNP treatment than when cells were treated with 10 μg/mL LPS, which served as a positive control, suggesting that the IRF3 pathway can be strongly induced by the expression of constitutively active cGAS.

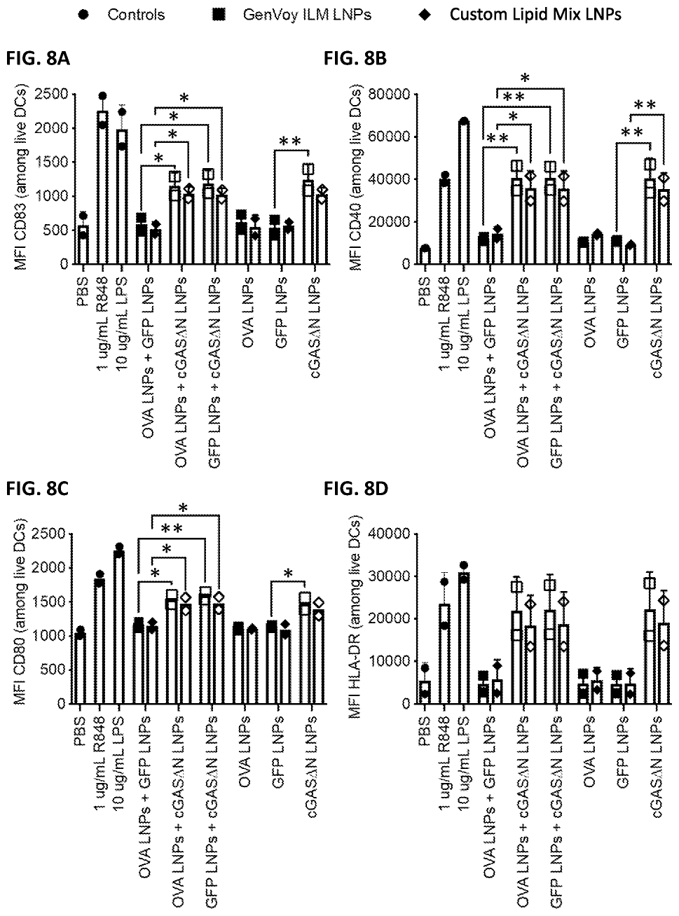

LNP-treated moDC were stained 1 day following treatment to quantify expression of activation markers. By measuring the median fluorescence intensity (MFI), CD83 expression increased when cells were treated with cGASΔN LNP compared to standard LNPs (FIG. 8A). Expression of T cell costimulatory molecules were also measured. CD40, CD80 expression increased in the overall population of moDC and also at the single cell level when they were treated with cGASΔN LNPs (FIG. 8B, FIG. 8C). While HLA-DR was expressed by nearly all moDC, MFI measurements revealed that MHC Class II molecules were increased on individual cells when moDC were treated with cGASΔN LNPs (FIG. 8D). In the case of MHC Class I molecules, HLA-ABC staining did not reveal any cGASΔN LNP-dependent changes.

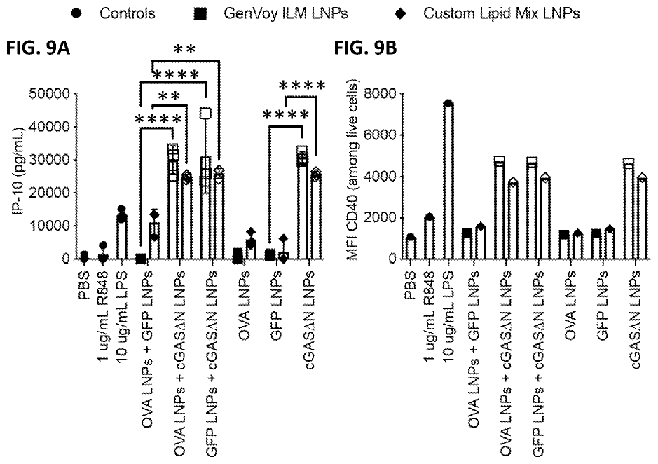

cGASΔNLNPs activate inflammatory pathways in THP-1. After one day of incubation, THP-1 cell supernatants were measured for the presence of cytokines that are dependent upon NF-kB or IRF3 activation, IL-6 and IP-10 respectively. At both time points, IL-6 was not differentially expressed by THP-1 cells treated with cGAS LNPs, suggesting that NF-kB is not activated. In contrast, IP-10, a gene controlled by IRF3, was expressed within a day of cGAS LNP treatment (FIG. 9A). cGAS LNPs induced IP-10 expression whereas other control LNPs induced little to no IP-10. The detectable IP-10 indicated that packaging a transcript of constitutively active cGAS into an LNP can be a method to activate the innate immune system.

To further verify that cGAS LNPs activate innate immune signaling, the THP-1 cells were collected 1 day after the start of incubation to stain for cell surface expression of activation markers. THP-1 samples were gated on live, single-cell events. CD40 expression was observed to increase after cell treatment with cGAS LNPs whereas other control LNPs carrying GFP or Ova transcripts did not (FIG. 9B). CD40 is a receptor that augments the immune response via cell-cell interactions with its cognate ligand.

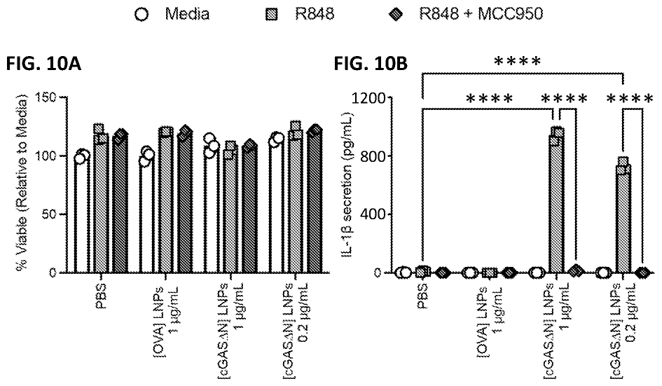

Treatment with cGASΔNLNPs+R848 results in moDC hyperactivation. moDCs were stimulated with R848 and cGASΔN to determine if the combination of NF-kB stimulation and cGAS-STING pathway activation would allow for more potent inflammatory activity. Specifically, the moDC's ability to be hyperactivated by this combination was tested. Hyperactivation was typified by the DCs' ability to secrete IL-1β while remaining viable. moDCs were either left unstimulated (PBS treated), or treated with cGASΔN LNPs or OVA LNPs in the presence of media, R848, or R848 and MCC950. MCC950 is an NLRP3 inflammasome inhibitor, so cells treated with MCC950 are not able to produce IL-1β in an inflammasome dependent manner. Importantly, all cells treated with LNPs showed similar viability to moDCs treated with PBS alone, indicating that none of the cells were pyroptosing in response to LNP treatment (FIG. 10A). To determine if any active IL-1β was produced by these cells in response to treatment with R848 and cGASΔN, supernatants from the moDC culture were added to HEK-Blue IL-1β reporter cells that produce SEAP in response to IL-13. As expected, moDCs treated with PBS, R848, or R848+MCC950 did not produce IL-1β (FIG. 10B). To determine if hyperactivation was dependent on the LNP lipid composition, LNPs containing OVA were cultured with R848—a condition which did not allow for IL-1β production. Interestingly, LNPs loaded with cGASΔN, when delivered in combination with R848, stimulated IL-1β secretion in moDC (FIG. 10B). This response was NLRP3 inflammasome dependent, as treatment with MCC950 eliminated IL-1β production.

Altogether, the data collected from treating THP-1 and human moDC with LNPs led to several conclusions. Firstly, the LNPs were successfully synthesized. Additionally, expression of constitutively active cGAS resulted in the upregulation of genes associated with the cGAS-STING pathway, in a formulation-independent manner. Inflammatory cytokines were expressed, and upregulation of activation markers were observed on the cell surface. Constitutively active cGAS encoded as mRNA and packaged in LNPs can be successfully utilized to initiate innate immune signaling in targeted cells to promote an inflammatory response. Lastly, constitutively active cGAS in combination with R848 allowed for moDC hyperactivation, with IL-1β secretion occurring in live DCs.

### Example 3: Murine Cells Are Activatable In Vivo By LNPs Containing cGASΔN mRNA

### Materials & Methods

Materials. Lipids for LNPs were purchased from Cayman Chemicals (SM102) or Avanti (22:0 LPC, DSPC, DMG-PEG2000). Cholesterol was purchased from Sigma. GenVoy ILM lipids were purchased from Precision Nanosystems. CleanCap OVA and GFP mRNA were purchased off-the-shelf from Trilink, with a 120 polyA tail and base modification 5-methoxyuridine, optimized for mammalian systems. cGASΔN mRNA was custom ordered and synthesized via in vitro transcription from a linearized template DNA (Trilink). The human cGASΔN sequence was codon optimized to be expressed in mouse. The sequence was capped using Trilink's proprietary Clean Cap mRNA technology, with base modification N1-methylpseudouridine and a 120 polyA tail. The sequence contains a Bbsl restriction enzyme site. The sequence information was used to build plasmids, from which mRNA was synthetically synthesized. mRNA was phosphatase treated after synthesis.

LNP Synthesis. Lipid nanoparticles (LNPs) were prepared as in Example 2 using the GenVoy ILM LNP lipid mix (Precision Nanosytems) or a typical LNP lipid mix (Table 2-1, Example 2). OVA mRNA, GFP mRNA, and cGASΔN mRNA (Trilink), were each prepared at 0.17 mg/mL in sodium citrate buffer, pH 4. GenVoy ILM and the typical LNP lipid mix were used at 12.5 mM. LNPs were synthesized using the NanoAssemblr Ignite instrument (Precision Nanosystems). Lipids in ethanol were combined with the mRNA solutions individually at a 1:3 volumetric ratio, using a flow rate of 12 mL/min. LNPs were washed in 10 volumes of phosphate buffered saline (PBS), pH 7.4 to remove residual ethanol, and then concentrated using Amicon 10K MWCO centrifugal filters. LNPs were filtered through a 0.2 μm filter before use.

LNP Characterization. Loading of mRNA into LNPs was quantified as in Example 2 using a RiboGreen assay (ThermoFisher) following the manufacturer's protocol. Samples were diluted to fall within the range of the standard curve. LNPs were lysed using Triton X-100 to assess encapsulation of mRNA into LNPs. Both total mRNA and encapsulated mRNA were quantified. The size of the LNPs was assessed using dynamic light scattering (DLS) on the NanoBrook Omni (Brookhaven). LNPs were diluted 1:10 in PBS before running on the DLS. Three 90 second measurements were recorded for each sample.

In vivo immunization with LNPs containing cGASΔNmRNA and OVA antigen mRNA. BL6 mice were immunized subcutaneously with LNPs following the table below (Table 3-1). Antigen was delivered using OVA mRNA-loaded LNPs, as well as LNPs containing either adjuvant cGASΔN mRNA or non-adjuvanting GFP mRNA. Mice were dosed with the mRNA doses indicated in the table. OVA mRNA dose was fixed at 5 μg/mouse, while adjuvant mRNA varied from 1 μg/mouse to 10 μg/mouse. Mice (n=5/group) were given a primary immunization on Day 0, with a boost immunization of the same doses on Day 7. 7 days post boost, blood was collected for antibody and T cell responses. Serum was collected from the blood using serum separation tubes, while blood for cellular analysis was collected using K2EDTA tubes. After blood collection, mice were euthanized and the draining lymph nodes and spleen were collected and processed to single cell suspension.

TABLE 3-1
LNPs used in in vivo assessment of T
and B cell responses to cGASΔN LNPs

| | mRNA encoding | mRNA encoding | |
|---|---|---|---|
| Group | OVA | GFP and/or cGASΔN | Formulation |
| 1 | None | None | PBS |
| 2 | None | GFP (5 μg/mouse) + | GV |
| | | cGASΔN (5 μg/mouse) | |
| 3 | OVA (5 μg/mouse) | GFP (1 μg/mouse) | GV |
| 4 | OVA (5 μg/mouse) | GFP (5 μg/mouse) | GV |
| 5 | OVA (5 μg/mouse) | cGASΔN (1 μg/mouse) | GV |
| 6 | OVA (5 μg/mouse) | cGASΔN (5 μg/mouse) | GV |
| 7 | OVA (5 μg/mouse) | GFP (5 μg/mouse) | Custom |
| 8 | OVA (5 μg/mouse) | cGASΔN (5 μg/mouse) | Custom |

OVA-specific T cell tetramer assessment. OVA-specific T cells in the blood of mice receiving OVA LNP immunization were assessed 7 days post boost. CD8+ T cells specific for SIINFEKL (SEQ ID NO:16), an OVA MHC-I epitope, were quantified in the blood using a tetramer analysis. Briefly, red blood cells in the blood were lysed using an RBC lysis buffer, with lysis completed twice to completely remove any RBCs in the blood. Cells were washed, then stained for viability (Live/Dead), SIINFEKL-tetramer binding (MBL), and CD3, CD4, and CD8 expression. Cells were fixed with 4% paraformaldehyde, and counting beads were added before running to allow for a total cell count. Data were collected using a BD FACS Symphony and analyzed using Flowjo (BD).

OVA-specific Antibody assessment. OVA-specific antibodies in the serum of mice receiving OVA LNP immunization were assessed 7 days post boost. OVA-specific total IgG, IgG1, and IgG2b were assessed using ELISA. Briefly, ELISA plates were coated with 10 g/mL Endofit Ovalbumin (Invivogen) overnight, then washed and blocked with 2% bovine serum albumin. Plates were washed again, and then serum was added to the plates at a 1:500 dilution, followed by 1:5 dilutions completed for a total of 7 serum dilutions tested. Samples were washed, then incubated with detection antibody specific for IgG, IgG1, or IgG2b conjugated to HRP (Southern Biotech), to detect total, Th2 or Th1 skewing OVA-specific antibodies respectively. Plates were washed, then incubated with TMB, and stop solution was added once color development was completed.

### Results

To determine if cGASΔN LNPs would induce greater antigen specific T and B cell responses in vivo, mice were immunized with OVA LNPs in combination with cGASΔN LNPs or GFP LNPs (Table 3-1), at different doses of cGASΔN or GFP mRNA. Mice were immunized and then boosted after 1 week. Blood was collected for antibody and T cell tetramer analysis 1 week after the boost. Two lipid formulations: GenVoy (Precision Nanosystems) and a custom lipid mix were included to determine if activity was LNP-formulation dependent.

cGASΔNLNPs exhibit similar sizing and loading profiles to LNPs loaded with model antigens. cGASΔN mRNA loaded LNPs were prepared according to Example 2, with characterization following the trends shown in FIG. 6A-6C. All LNPs showed similar size and mRNA loading profiles to the LNPs loaded with model antigen mRNAs—OVA and GFP. All mRNA-loaded LNPs had an average effective diameter less than 150 nm, with a relatively uniform size profile, exhibited by polydispersity indexes less than 0.3. All mRNAs loaded into the GenVoy TLM LNPs with 70-90% of the theoretically loaded amount of mRNA encapsulated in the LNPs.

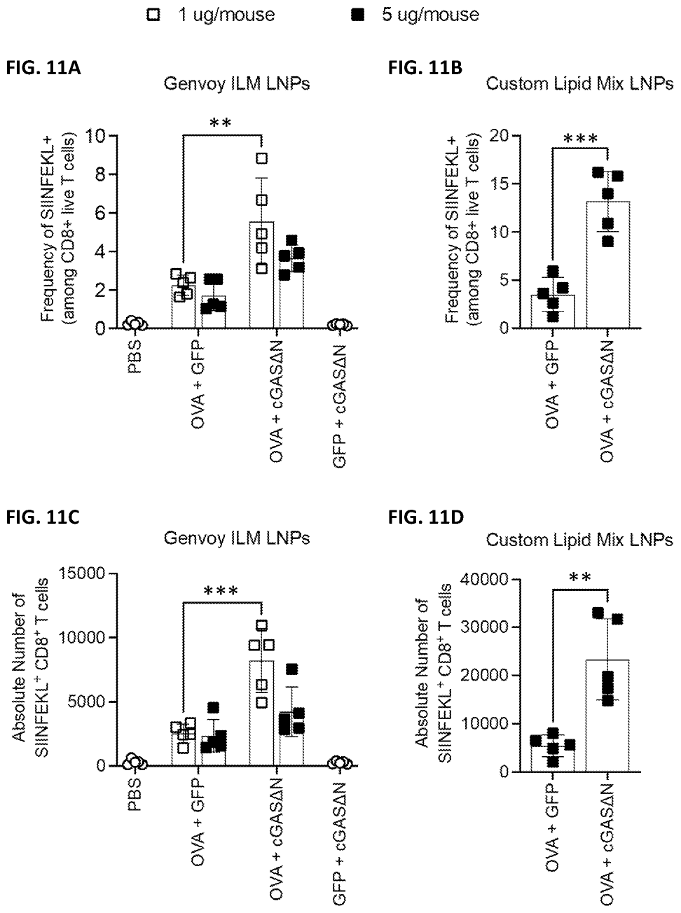

Treatment with OVA LNPs and cGASΔNLNPs increases OVA-specific CD8+ T cells. After two immunizations with OVA LNPs (5 μg mRNA/mouse) delivered with cGASΔN LNPs (1 or 5 μg mRNA/mouse) or control GFP LNPs (1 or 5 μg mRNA/mouse) (Table 3-1), OVA-specific T cell presence in the blood was analyzed. Blood was collected 7 days post-boost, processed to remove red blood cells, and then stained for Live/Dead, CD3, CD8, CD4, and SIINFEKL-tetramer reactivity (OVA-specific CD8+ T cell epitope set forth as SEQ ID NO:16). Frequency of SIINFEKL-specific cells among live CD8+ T cells was determined, as well as the total absolute number of SIINFEKL-specific CD8+ T cells collected in the blood. When mice were treated with the GenVoy LNPs—OVA LNPs (5 μg mRNA), with cGASΔN or GFP LNPs (1 or 5 μg mRNA), the frequency of SIINFEKL-specific (OVA-specific) CD8+ T cells significantly increased when cGASΔN mRNA was delivered in addition to OVA, compared to when GFP mRNA was delivered in addition to OVA (FIG. 11A). Mice that did not receive OVA LNPs did not have SIINFEKL-specific CD8+ T cells. This trend was not formulation specific, as mice that received OVA LNPs (5 μg mRNA) and cGASΔN (5 μg mRNA) prepared with the custom lipid mix had a significantly higher frequency of SIINFEKL-specific T cells in the blood than mice that received OVA LNPs+GFP LNPs (5 μg mRNA) (FIG. 11B). This trend also held true for the absolute number of SIINFEKL-specific CD8+ T cells, when prepared with either the GenVoy (FIG. 11C) or custom lipid mix (FIG. 11D). Interestingly, lowering the dose of cGASΔN mRNA did not seem to have a big impact on the frequency of OVA-specific T cells, suggesting that small amounts of this cGASΔN mRNA, in combination with standard lipid preparations containing antigen mRNA may be sufficient to significantly boost an antigen-specific T cell response.

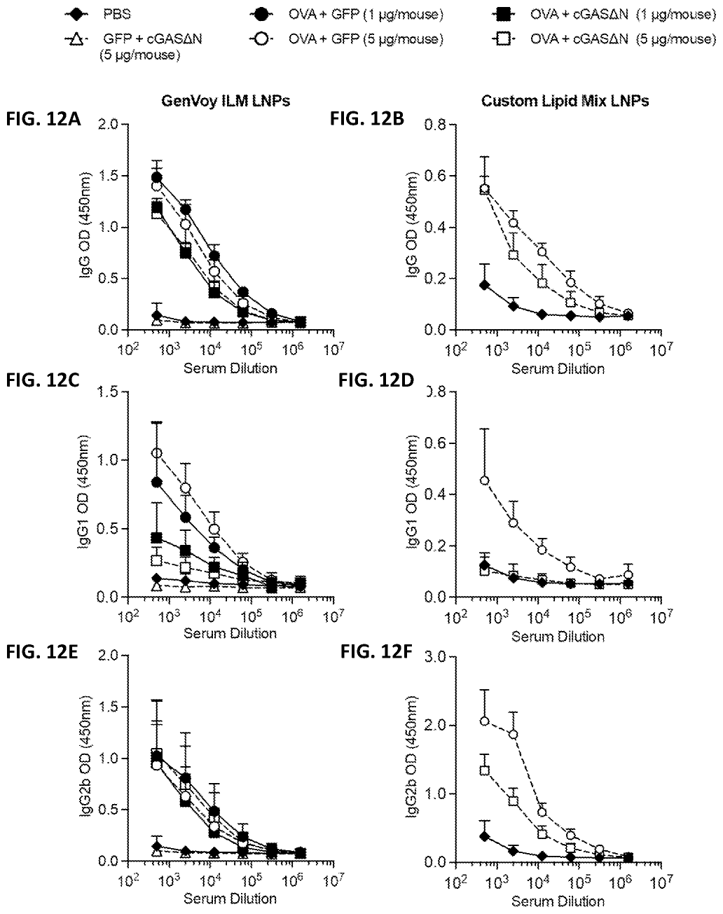

Treatment with cGASΔNLNPs decreases Th2 specific OVA-specific antibodies without impacting Th1 specific IgG responses. After two immunizations with OVA LNPs (5 μg mRNA/mouse) delivered with cGASΔN LNPs (1 or 5 μg mRNA/mouse) or control GFP LNPs (1 or 5 μg mRNA/mouse) (Table 3-1), OVA-specific antibodies in the blood were quantified. Blood was collected 7 days post-boost, serum was isolated, after which total IgG, IgG1 (associated with Th2 responses), and IgG2a (associated with Th1 responses), were analyzed. Traditionally, immunization with mRNA antigen loaded LNPs results in strong antibody responses, so antibody response was assessed to determine if the addition of cGASΔN LNPs in the treatment would reduce the expected response. Interestingly, mice treated with OVA and cGASΔN LNPs prepared using the GenVoy formulations did not show significant changes in OVA-specific total IgG measured compared to OVA+GFP LNP treatment (FIG. 12A). Mice that did not receive OVA LNPs did not produce OVA-specific antibodies. This was not a formulation specific response: OVA-specific total IgG measured was not significantly impacted by the presence cGASΔN in LNPs prepared using the custom lipid mix (FIG. 12B). OVA-specific IgG1 was also assessed, as IgG1 is typically associated with a Th2 immune responses. OVA-specific IgG1 was significantly reduced by the presence cGASΔN in LNPs prepared using GenVoy formulations (p<0.05 for the 1:500 and 1:2500 dilution at both 1 μg and 5 ug cGASΔN vs GFP LNPs) (FIG. 12C). This reduced OVA-specific IgG1 responses was also measured in mice treated with cGASΔN LNPs prepared using the custom lipid mix (p<0.05 for the 1:500, 1:2500, and 1:12500 dilution at 5 ug cGASΔN vs GFP LNPs) (FIG. 12D). IgG2a levels were also assessed, as IgG2a is associated with more strongly inflammatory Th1 responses. Importantly, mice treated with OVA and cGASΔN LNPs prepared using the GenVoy formulations (FIG. 12E) or the custom lipid mix (FIG. 12F) did not show significant changes in OVA-specific IgG2a compared to OVA+GFP treated mice.

Taken together, the data collected from the in vivo immunization experiment show that treating mice with cGASΔN LNPs in combination with antigen LNPs significantly improves antigen specific adaptive immune responses. Mice receiving cGASΔN LNPs showed significant increases in the number of antigen-specific T cells, without sacrificing antibody responses. In fact, the decrease in antigen-specific IgG1 without impacting the total antigen-specific IgG levels shows a skewing toward a Th1 associated immune response, which is beneficial in inducing protective immune responses (which are needed for immunotherapies). Combined, these data suggest that adding a low dose of cGASΔN LNPs to a standard antigen LNP preparation could result in much more potent immune responses to LNP vaccines.

SEQUENCES

SEQ ID NO: 1
>HUMAN
PGASKLRAVLEKLKLSRDDISTAAGMVKGVVDHLLLRLKCDSAFRGVGLLN
TGSYYEHVKISAPNEFDVMFKLEVPRIQLEEYSNTRAYYFVKFKRNPKENPLSQFLEGEILSASKMLSKF
RKIIKEEINDIKDTDVIMKRKRGGSPAVTLLISEKISVDITLALESKSSWPASTQEGLRIQNWLSAKVRK
QLRLKPFYLVPKHAKEGNGFQEETWRLSFSHIEKEILNNHGKSKTCCENKEEKCCRKDCLKLMKYLLEQL
KEREKDKKHLDKFSSYHVKTAFFHVCTQNPQDSQWDRKDLGLCFDNCVTYFLQCLRTEKLENYFIPEFNL
FSSNLIDKRSKEFLTKQIEYERNNEFPVFDEF
SEQ ID NO: 2
>M._MULATTA
PGARKLRAVLEKLRLSHQDISKAAKVVNGVVGHLLRRLKCEPEFGGVEQLH
TGSYYEHVKISAPNEFDVMFKLQVPRIQLEEYSDTGAYYFVKFKRNPEGNPLSLFLEDEILSASKMLSKF
RKIIKEEINNIKDVQPDADVIMKRKRPGSPAVTLLINKEISVDITLALELKTSWPASTQEGLAIKNWLSA
KVRQQLRLKPFYLVPKHAKEGNGFQEETWRLSFSHIEKEILSNHGKSKTCCENKEMKCCRKDCLKLMKYL
LEQLKEKFKDKKHLDKFSSYHVKTAFFHLCTENPQDSQWDPKDLGLCFDNCVTYFLECLRTEQLKNYFIP
GFNLFSSNLIDKTSKEFLSKQIEYERNNEFPVFGEF
SEQ ID NO: 3
>P._ANUBIS
PGARKLRAVLEKLRLSHQDISKAAKVVNGVVGHLLHRLKCESEFEGVEQLH
TGSYYEHVKISAPNEFDVMFKLQVPRIQLEEYSDTGAYYFVKFKRNPKGNPLSQFLEDEILSASKMLSQF
RKIIKEEINNIKDTDVIMKRKRRGSPAVTLLINKEISVDITLALELKTSWPASTQEGLAIKNWLSAKVRQ
QLRLKPFYLVPKHAKEGNGFQEETWRLSFSHIEKEILSNHGKSKTCCENKEMKCCRKDCLKLMKYLLEQL
KEKFKDKKHLDKFSSYHVKTAFFHLCTENPQDSQWDPKDLGLCFDNCVTHFLQCLRTEKLANYFIPGFNL
FSSNLIDKSSKEFLSKQIEYERNNEFPVFGEF
SEQ ID NO: 4
>N._LEUCOGENYS
PGARKLRAVLEKLKLSRQEISEAAEVVNGVVDHLLRRLOKCDSEFRGVGLLRTGSYYEH
VKISAPNEFDVMFKLEVPRIQLEEYSNTGAYYFVKFKRNPKENPLSQFLEGEILSASKMLSKFRKIIKEE
INNIKDTDVIMKRKRGGSPAVTLLISEKISVDITLALESNSNSWPASTQEGLPIKDWLSGKVRRQLRLKP
FYLVPKHAKEGNGFQEETWRLSFSHIEKEILNNHGKTKTCCENKNVKCCRKDCLKLMKYLLEQLKERFKD
KKHLDKFSSYHVKTAFFHVCTENPQDSQWDPKNLGFCFDKCVIYFLKCLRTEQLGNYFIPGFNLFSSNLI
DKRSKEFLSKQIEYERNNEYPVFG
SEQ ID NO: 5
>H_LAR
PGARKLPAVLEKLKLSRQ
EICEAAEVVNGVVDHLLRRLQKCDSEFRGVGLLRTGSYYEHVKISAPNEFDVMFKLEVPRIQLEEYSNTG
AYYFVKFKRNPKENPLCQFLEGEILSASKMLSKFRKIIKEEINNIKDTDVILKRKRGGSPAVTLLISEKI
SVDITLALESKSSSWPASTQEGLPIKDWLSGKVRTQLRLKPFYLVPKHAKEGNGFQEETWRLSFSHIEKE
ILNNHGETKTCCENKKVKCCRKDCLKLMKYLLEQLKERFKDKKHLDKFSSYHVKTAFFHVCTENPKDSQW
DPKDLGLCFDKCVIYFLQCLSTEQLGNYFIPGFNLESSNLIDKRSKEFLSKQIEYERNNEYPVFGKC
SEQ ID NO: 6
>P._ABELII
PGASKLRAVLEKLKLSRLEISKAAEVVNRVVDHLLRRLQKYNSEFRGVGLLRT
GSYYEHVKISAPNEFDVMFKLEVPRIQLEEYSNTGAYYFVKFKRNPKENPLSQFLEGEILSASKMLSKFR
KIIKEEINNIKDTDVIMKRKRGGSPAVTLLISEEISVDITLALESKSSWPASTQEGLPIKNWLSAKVRKQ
LRLKPFYLVPKHAKEGNGFQEETWRLSFSHIEKEILNNHGKSKTCCEDKEVKCCRKDCLKLMKYLLEQLK
ERFKDKKHLDKFSSYHVKTAFFHVCTQNPQDSQWDPKDLGLCFDNCVTYFLQCLRTEQLGNYFIPGFNLF
SSNLIDKRSKEFLTKQIEYERNNEFPVFDEF
SEQ ID NO: 7
>P._TROGLODYTES
PGASKLRAVLEKLKLSRDDISTAAGMVKGVVDHLLLRLKCDSAFRGVGLLN
TGSYYEHVKISAPNEFDVMFKLEVPRIQLEEYSNTRAYYFVKFKRNPKENPLSQFLEGEILSASKMLSKF
RKIIKEEINNIKDVIMKRKRGGSPAVTLLISEKISVDITLALESKSSWPASTQEGLPIKNWLSAKVRKQL
RLKPFYLVPKHAKEGNGFQEETWRLSFSHIEKEILNNHGKSKTCCEIKEAKCCRKDCLKLMKYLLEQLKE
KFKDKKHLDKFSSYHVKTAFFHVCTENPQDSQWDRKDLGLCFDNCVTYFLQCLRIEKLENYFIPEFNLFS
SDLIDKRSKEFLTKQIEYERNNEFPVFDEF
SEQ ID NO: 8
>G._GORILLA
PGASKLRAVLEKLKLSRDDISTAAGMVKGVVDHLLLRLKCDSAFRGVGLLN
TGSYYEHVKISAPNEFDVMFKLEVPRIQLEEYSNTRAYYFVKFKRNPKENPLSQFLEGEILSASKMLSKF
RKIIKEEINNIKDTDVIMKRKRGGSPAVTLLISEKISVDITLALESKSSWPASTQEGLRIQNWLSAKVRR
QLRLKPFYLVPKHAKEGNGFQEETWRLSFSHIEKEILNNHGKSKTCCENKEAKCCRKDCLKLMKYLLEQL
KERFKDKKHLDKFSSYHVKTAFFHVCTQNPQDSQWDRKQLGLCFDNCVTYFLQCLRTERLENYFIPEFNL
FSRNLIDKRSKEFLTKQIEYERNNEFPVFDEF
SEQ ID NO: 9
>CGASΔN_CONSENSUS
PGAXKLXAVLEKLXLSXXXIXXAAXXVXXVVXHLLXRLXXXXXXFXGVXXLX
TGSYYEHVKISAPNEFDVMFKLXVPRIQLEEYSXTXAYYFVKFKRNPXXNPLXXFLEXEILSASKMLSXF
RKIIKEEINXIKXXXXXXDVIXKRKRXGSPAVTLLIXXXISVDITLALEXXXXSWPASTQEGLXIXXWLS
XKVRXQLRLKPFYLVPKHAKEGNGFQEETWRLSFSHIEKEILXNHGXXKTCCEXKXXKCCRKDCLKLMKY
LLEQLKEXFKDKKHLDKFSSYHVKTAFFHXCTXNPXDSQWDXKXLGXCFDXCVTXFLXCLXXEXLXNYFI
PXFNLFSXXLIDKXSKEFLXKQIEYERNNEXPVFXXX
SEQ ID NO: 10
>HUMANCGAS_FULL-LENGTH
MQPWHGKAMQRASEAGATAPKASARNARGAPMDPTESPAAPEAALPKAGKFGPARKSGSRQKKSAPDTQE
RPPVRATGARAKKAPQRAQDTQPSDATSAPGAEGLEPPAAREPALSRAGSCRQRGARCSTKPRPPPGPWD
VPSPGLPVSAPILVRRDAAPGASKLRAVLEKLKLSRDDISTAAGMVKGVVDHLLLRLKCDSAFRGVGLLN
TGSYYEHVKISAPNEFDVMFKLEVPRIQLEEYSNTRAYYFVKFKRNPKENPLSQFLEGEILSASKMLSKF
RKIIKEEINDIKDTDVIMKRKRGGSPAVTLLISEKISVDITLALESKSSWPASTQEGLRIQNWLSAKVRK
QLRLKPFYLVPKHAKEGNGFQEETWRLSFSHIEKEILNNHGKSKTCCENKEEKCCRKDCLKLMKYLLEQL
KERFKDKKHLDKFSSYHVKTAFFHVCTQNPQDSQWDRKDLGLCFDNCVTYFLQCLRTEKLENYFIPEFNL
FSSNLIDKRSKEFLTKQIEYERNNEFPVFDEF
SEQ ID NO: 11
>HUMANCGAS_N-TERMINUS
MQPWHGKAMQRASEAGATAPKASARNARGAPMDPTESPAAPEAALPKAGKFGPARKSGSRQKKSAPDTQE
RPPVRATGARAKKAPQRAQDTQPSDATSAPGAEGLEPPAAREPALSRAGSCRQRGARCSTKPRPPPGPWD
VPSPGLPVSAPILVRRDAA
SEQ ID NO: 12
>THOSEA ASIGNA VIRUS T2A RIBOSOME-SKIPPING PEPTIDE
[GSG]EGRGSLLTCGDVEENPGP
SEQ ID NO: 13
>PORCINE TESCHOVIRUS-1 P2A RIBOSOME-SKIPPING PEPTIDE
[GSG]ATNFSLLKQAGDVEENPGP
SEQ ID NO: 14
>EQUINE RHINITIS A VIRUS E2A RIBOSOME-SKIPPING PEPTIDE
[GSG]QCTNYALLKLAGDVESNPGP
SEQ ID NO: 15
>FOOT-AND-MOUTH DISEASE VIRUS F2A RIBOSOME-SKIPPING PEPTIDE
[GSG]VKQTLNFDLLKLAGDVESNPGP
SEQ ID NO: 16
>GALLUS GALLUS OVALBUMIN PEPTIDE
SIINFEKL
SEQ ID NO: 17
>SYNTHETIC CGASAN
ATGCCCGGCGCCAGCAAGCTGAGGGCCGTGCTGGAGAAGCTGAAGCTGAGCAGGGACGACATCA
GCACCGCCGCCGGCATGGTGAAGGGCGTGGTGGACCACCTGCTGCTGAGGCTGAAGTGCGACAG
CGCCTTCAGGGGCGTGGGCCTGCTGAACACCGGCAGCTACTACGAGCACGTGAAGATCAGCGCC
CCCAACGAGTTCGACGTGATGTTCAAGCTGGAGGTGCCCAGGATCCAGCTGGAGGAGTACAGCA
ACACCAGGGCCTACTACTTCGTGAAGTTCAAGAGGAACCCCAAGGAGAACCCCCTGAGCCAGTT
CCTGGAGGGCGAGATCCTGAGCGCCAGCAAGATGCTGAGCAAGTTCAGGAAGATCATCAAGGAG
GAGATCAACGACATCAAGGACACCGACGTGATCATGAAGAGGAAGAGGGGCGGCAGCCCCGCCG
TGACCCTGCTGATCAGCGAGAAGATCAGCGTGGACATCACCCTGGCCCTGGAGAGCAAGAGCAG
CTGGCCCGCCAGCACCCAGGAGGGCCTGAGGATCCAGAACTGGCTGAGCGCCAAGGTGAGGAAG
CAGCTGAGGCTGAAGCCCTTCTACCTGGTGCCCAAGCACGCCAAGGAGGGCAACGGCTTCCAGG
AGGAGACCTGGAGGCTGAGCTTCAGCCACATCGAGAAGGAGATCCTGAACAACCACGGCAAGAG
CAAGACCTGCTGCGAGAACAAGGAGGAGAAGTGCTGCAGGAAGGACTGCCTGAAGCTGATGAAG
TACCTGCTGGAGCAGCTGAAGGAGAGGTTCAAGGACAAGAAGCACCTGGACAAGTTCAGCAGCT
ACCACGTGAAGACCGCCTTCTTCCACGTGTGCACCCAGAACCCCCAGGACAGCCAGTGGGACAG
GAAGGACCTGGGCCTGTGCTTCGACAACTGCGTGACCTACTTCCTGCAGTGCCTGAGGACCGAG
AAGCTGGAGAACTACTTCATCCCCGAGTTCAACCTGTTCAGCAGCAACCTGATCGACAAGAGGA
GCAAGGAGTTCCTGACCAAGCAGATCGAGTACGAGAGGAACAACGAGTTCCCCGTGTTCGACGA
GTTCTAATGA

We claim:

1. A composition comprising an mRNA encapsulated in a lipid nanoparticle (LNP), wherein the mRNA comprises a coding region of a constitutively active cyclic GMP-AMP synthase (cGAS), and the LNP comprises a phospholipid, and at least one lipid selected from the group consisting of an ionizable lipid, a pegylated lipid, a structural lipid, and mixtures thereof.

2. A composition comprising a first mRNA and a second mRNA encapsulated in a lipid nanoparticle (LNP), wherein the first mRNA comprises a coding region of a constitutively active cyclic GMP-AMP synthase (cGAS), the second mRNA comprises a coding region of an antigen; and the LNP comprises a phospholipid, and at least one lipid selected from the group consisting of an ionizable lipid, a pegylated lipid, a structural lipid, and mixtures thereof.

3. A composition comprising an mRNA encapsulated in a lipid nanoparticle (LNP), wherein the mRNA comprises a first coding region and a second coding region separated by a 2A-like sequence, wherein the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the LNP comprises a phospholipid, and at least one lipid selected from the group consisting of an ionizable lipid, a pegylated lipid, a structural lipid, and mixtures thereof.

4. The composition of any one of claims 1-3, wherein the at least one lipid comprises an ionizable lipid, a pegylated lipid, and a structural lipid.

5. The composition of any one of claims 1-4, wherein the ionizable lipid comprises:
i) 8-[(2-hydroxyethyl)[6-oxo-6-(undecyloxy)hexyl]amino]-octanoic acid, 1-octylnonyl ester (SM-102) or analogs or derivatives thereof; and/or
ii) 6-((2-hexyldecanoyl)oxy)-N-(6-((2-hexyldecanoyl)oxy)hexyl)-N-(4-hydroxybutyl)hexan-1-aminium (ALC-0315) or analogs or derivatives thereof, and/or
iii) (6Z,9Z,28Z,31Z)-heptatriaconta-6,9,28,31-tetraen-19-yl 4-(dimethylamino)butanoate (DLin-MC3-DMA) or analogs or derivatives thereof.

6. The composition of any one of claims 1-5, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

7. The composition of any one of claims 1-5, wherein the pegylated lipid comprises polyethylene glycol [PEG]2000 dimyristoyl glycerol [DMG].

8. The composition of any one of claims 1-7, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

9. The composition of any one of claims 1-7, wherein the structural lipid comprises cholesterol.

10. The composition of any one of claims 1-9, wherein the phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

11. The composition of any one of claims 1-9, wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanola mine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

12. The composition of claim 11, wherein the phospholipid comprises 1,2-distearoyl-sn-glycero-3-phosphocholine (DSPC).

13. The composition of any one of claims 1-12, wherein the composition further comprises at least one excipient, optionally wherein the excipient comprises sucrose.

14. The composition of any one of claims 1-13, wherein the composition does not comprise a lysophosphatidylcholine (LPC), optionally wherein the LPC has a single C13-C24 acyl chain.

15. A composition comprising:
an mRNA complexed with one or more lipids (RNA-Lipoplex); and
wherein the mRNA comprises a coding region of a constitutively active cyclic GMP-AMP synthase (cGAS), and the one or more lipids comprise a first lipid and a second lipid.

16. A composition comprising:
a first mRNA and a second mRNA complexed with one or more lipids (RNA-Lipoplex); and
wherein the first mRNA comprises a coding region of a constitutively active cyclic GMP-AMP synthase (cGAS), and the second mRNA comprises a coding region of an antigen, and the one or more lipids comprise a first lipid and a second lipid.

17. A composition comprising:
an mRNA complexed with one or more lipids (RNA-Lipoplex); and
wherein the mRNA comprises a first coding region and a second coding region separated by a 2A-like sequence, the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the one or more lipids comprise a first lipid and a second lipid.

18. The composition of any one of claims 15-17, wherein the first lipid is a cationic lipid, and the second lipid is a neutral or anionic lipid.

19. The composition of claim 18, wherein the cationic lipid comprises one or both of:
i) 1,2-di-O-octadecenyl-3-trimethylammonium propane (DOTMA) or analogs or derivatives thereof; and
ii) 1,2-dioleoyl-3-trimethylammonium propane (DOTAP) or analogs or derivatives thereof.

20. The composition of claim 18 or claim 19, wherein the neutral or anionic lipid comprises:
i) 1,2-di-(9Z-octadecenoyl)-sn-glycero-3-phosphoethanolamine (DOPE) or analogs or derivatives thereof; and/or
ii) cholesterol or analogs or derivatives thereof, and/or
iii) 1,2-dioleoyl-sn-glycero-3-phosphocholine (DOPC) or analogs or derivatives thereof.

21. The composition of any one of claims 1-20, further comprising a TLR7/8 agonist.

22. The composition of claim 21, wherein the TLR7/8 agonist is a small molecule with a molecule weight of 900 daltons or less.

23. The composition of claim 22, wherein the TLR7/8 agonist comprises an imidazoquinoline compound.

24. The composition of claim 23, wherein the TLR7/8 agonist comprises resiquimod (R848).

25. The composition of any one of claims 1-24, wherein the mRNA or the first mRNA and the second mRNA comprises a 5′ untranslated region (5′UTR) and a 3′ untranslated region (3′UTR).

26. The composition of any one of claims 1-25, wherein the mRNA comprises a 5′ cap structure.

27. The composition of any one of claims 1-26, wherein the mRNA comprises a polyA tail.

28. The composition of any one of claims 1-27, wherein the mRNA is a nucleoside-modified mRNA.

29. The composition of any one of claims 1-28, wherein the constitutively-active cGAS has a greater propensity to self DNA reactivity than its wild-type counterpart.

30. The composition of any one of claims 1-29, wherein the cGAS is a truncated cGAS devoid of an amino-terminal phosphoinositide-binding domain (cGASΔN).

31. The composition of claim 30, wherein the cGASΔN comprises the amino acid sequence of SEQ ID NO:1 or the amino acid sequence at least 85%, 90%, 91%, 92%, 93%, 94%, 95%, 96%, 97%, 98%, or 99% identical to SEQ ID NO:1.

32. The composition of claim 31, wherein the cGASΔN comprises:
(i) the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8; or
(ii) the consensus amino acid sequence of SEQ ID NO:9.

33. The composition of claim 31, wherein the cGASΔN is encoded by the nucleotide sequence of SEQ ID NO:17.

34. The composition of any one of claims 30-33, wherein the coding region of the cGASΔN is in operable combination with a start codon (ATG).

35. The composition of any one of claims 1-34, wherein the antigen is a tumor antigen.

36. The composition of claim 35, wherein the tumor antigen is a tumor-associated antigen.

37. The composition of claim 35, wherein the tumor antigen is a neoantigen.

38. The composition of any one of claims 1-34, wherein the antigen comprises a microbial antigen.

39. The composition of claim 38, wherein the microbial antigen comprises a viral antigen, a bacterial antigen, a protozoan antigen, or a fungal antigen.

40. The composition of any one of claims 1-34, wherein the antigen comprises a surface protein or fragment thereof of a pathogen.

41. The composition of claim 40, wherein the pathogen is capable of causing disease in human subjects.

42. The composition of claim 40 or claim 41, wherein the pathogen is a virus.

43. The composition of claim 42, wherein the virus is a SARS-CoV-2.

44. The composition of claim 43, wherein the antigen is a spike (S) glycoprotein of the SARS-CoV-2, optionally wherein the spike glycoprotein is a pre-fusion stabilized variant.

45. The composition of any one of claims 1-44, wherein the composition does not comprise lipopolysaccharide (LPS) or monophosphoryl lipid A (MPLA).

46. The composition of any one of claims 1-45, wherein the composition does not comprise oxidized 1-palmitoyl-2-arachidonoyl-sn-glycero-3-phosphorylcholine (oxPAPC) or a species of oxPAPC.

47. The composition of claim 46, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).

48. A pharmaceutical formulation comprising the composition of any one of claims 1-47, and a pharmaceutically acceptable excipient.

49. A method for production of hyperactivated dendritic cells, the method comprising contacting the dendritic cells with an effective amount of the composition of any one of claims 1-47, any one of claims 21-47, or the formulation of claim 48 to produce hyperactivated dendritic cells, wherein the hyperactivated dendritic cells secrete IL-1beta without undergoing cell death within about 48 hours of exposure.

50. The method of claim 49, wherein the dendritic cells are:
(i) contacted in vivo with the composition; or
(ii) contacted ex vivo with the composition.

51. The method of claim 49 or claim 50, wherein the hyperactivated dendritic cells:
(i) secrete higher levels of one or more of RANTES, IP-10 and IFNα than unstimulated dendritic cells or dendritic cells contacted with empty LNPs; and/or
(ii) express higher levels of at least one cell surface marker selected from the group consisting of CD40, CD86, CD69, MHC class II, MHC class I, CCR7, and combinations thereof.

52. A pharmaceutical formulation comprising at least 10\hat{ }3, 10\hat{ }4, 10\hat{ }5 or 10\hat{ }6 of the hyperactivated dendritic cells produced by the method of any one of claims 49-51, and a pharmaceutically acceptable excipient.

53. A method of stimulating an immune response against an antigen, comprising administering an effective amount of the pharmaceutical formulation of claim 48 or claim 52 to an individual in need thereof to stimulate the immune response against the antigen.

54. A method of treating cancer, comprising administering an effective amount of the pharmaceutical formulation of claim 48 or claim 52 to an individual in need thereof to treat the cancer.

55. A method of inhibiting abnormal cell proliferation, comprising administering an effective amount of the pharmaceutical formulation of claim 48 or claim 52 to an individual in need thereof to inhibit abnormal cell proliferation.

56. A method of treating or preventing an infectious disease, comprising administering an effective amount of the pharmaceutical formulation of claim 48 to an individual in need thereof to treat or prevent the infectious disease.

57. The method of claim 56, wherein the infectious disease is a viral disease.

58. The method of claim 56, wherein the infectious disease is a bacterial disease.

59. The method or pharmaceutical formulation of any one of claims 49-55, wherein the dendritic cells are mammalian cells.

60. The method or pharmaceutical formulation of claim 59, wherein the mammalian cells are human cells.

61. The method of any one of claims 53-59, wherein the individual is mammal.

62. The method of claim 61, wherein the mammal is a human.

63. The method of claim 61, wherein the mammal is a dog or a cat.

64. The composition, formulation, or method of any one of claims 1-63, wherein the phospholipid comprises 1,2-distearoyl-sn-glycero-3-phosphocholine (DSPC), and the least one lipid comprises 8-[(2-hydroxyethyl)[6-oxo-6-(undecyloxy)hexyl]amino]-octanoic acid, 1-octylnonyl ester (SM-102) or analogs or derivatives thereof, and cholesterol.

65. The composition, formulation, or method of claim 64, wherein the at least one lipid further comprises a pegylated lipid, optionally wherein the pegylated lipid comprises polyethylene glycol [PEG]2000 dimyristoyl glycerol [DMG].

66. The composition, formulation, or method of any one of claims 1-65, wherein the LNP has an effective diameter of less than about 250 nanometers, optionally from about 25 to about 250 nanometers, optionally from about 50 to about 200 nanometers, or optionally from about 75 to about 175 nanometers.

67. The composition, formulation, or method of claim 66, wherein the LNP has an effective diameter of less than about 200 nanometers.

68. The composition, formulation, or method of claim 67, wherein the LNP has an effective diameter of less than about 150 nanometers.
